(19) United States
(12) Patent Application Publication
(10) Pub. No.: US 2016/0256677 A1
(43) Pub. Date: Sep. 8, 2016

(54) FLEXIBLE DIGITAL IMAGE SENSOR

(71) Applicant: The Board of Trustees of the University of Alabama, Tuscaloosa, AL (US)

(72) Inventors: Jinhui Song, Northport, AL (US); Chengming Jiang, Tuscaloosa, AL (US)

(21) Appl. No.: 15/058,528

(22) Filed: Mar. 2, 2016

Related U.S. Application Data
(60) Provisional application No. 62/126,970, filed on Mar. 2, 2015.

(51) Int. Cl. A61N 1/05; H01L 31/0296; H01L 31/18; H01L 31/108; H01L 31/0224; H01L 27/146

(52) U.S. Cl. CPC A61N 1/0543; H01L 31/022408; H01L 27/14601; H01L 31/022466; H01L 31/1884; H01L 27/14683; H01L 31/108; H01L 31/0296; H01L 27/14618

(57) ABSTRACT

Systems and methods are disclosed that describe flexible photo-sensing pixels and interconnects that have comparable pixel densities and functionality as the human retina. The pixels comprise vertically aligned, nanowire cluster piles that serve as the three-dimensionally compressible photoreceptor pixels, and flexible, transparent interconnected electrodes. Shape-adaptive high-resolution optic-electrical imaging system are described that can serve as a human retina and a retinal prosthesis for restoring vision.

This application claims priority to, and the benefit of, U.S. Provisional Application No. 62/126,970, filed Mar. 2, 2015, titled “Flexible Digital Image Sensor,” the text of which is incorporated by reference herein in its entirety.

### BACKGROUND

Generally, existing silicon-based technologies lead to relatively large and rigid thin-film photo-sensing pixels. This can lead to challenges when such devices are to be utilized in applications where a conformable shape, small size and high resolution is desired such as, for example, a biomimetic retinal implant.

Biomimetic prostheses seek to replace human tissues/organs and restore functionality. The human retina has nearly one hundred thousand photoreceptor cells in a millimeter-size soft tissue and is central to vision in the eye system. Retinal prostheses seeks to mimic the human retina in material, mechanics, and morphologies. Unfortunately, existing technologies for retinal prostheses have a limited number of micro-metal electrodes implanted, leaving bulky parts that sense vision information outside the body. Image sensing techniques can be affected by pixel size and pixel deformability. Implantable electronic image sensors with the same hemispherical shape as a human retinal prostheses have many design and fabrication considerations. As noted above, existing silicon-based technologies lead to relatively large and rigid thin-film photo-sensing pixels. Retinal prostheses with comparable resolution to the eye and flexibility in three dimensions to conform to the lining tissue in a human eyeball are lacking.

Therefore, what are needed are devices, systems and methods that overcome challenges in the present art, some of which are described above.

### SUMMARY

In an aspect of this disclosure, a device comprising flexible photo-sensing pixels is described. In one exemplary application, embodiments of such a device can be integrated with comparable pixel densities and functionality as the human retina. In one aspect of this disclosure, the pixels comprise vertically aligned, nanowire cluster piles that serve as the three-dimensionally compressible photoreceptor pixels, and flexible, transparent interconnected electrodes. The material chosen for the device can result in the device being transformable onto any curvilinear surface. Embodiments of the device can realize shape-adaptive high-resolution optic-electrical imaging system such as, for example, a human retina and in one exemplary application of the technology can serve as a retinal prosthesis for restoring vision for patients with degenerative retinal diseases.

In another aspect of the disclosure, an optical device comprising semiconducting material sandwiched by two electrodes is disclosed. In an exemplary application of the optical device, it can at least partially comprise a retinal implant.

The optical device can at least partially comprise biocompatible materials. It can at least be partially comprised of translucent materials. It can at least partially be comprised of materials that allow light to pass through them. It can be deformable. The semiconducting material of the device can exhibit a photon effect. The semiconducting material can comprise material that exhibits a photo-resistance change under illumination. The semiconducting material can comprise nanowires. The nanowires can be arranged in an array pattern. The nanowires can be at least partially comprised of zinc oxide. The optical device can have the zinc oxide deposited through sputtering. The nanowire array can comprise at least one pixel. The at least one pixel can comprise individually clustered groups of the nanowires. In one aspect, at least one of the two electrodes comprises semi-transparent semiconducting material. In another aspect, at least one of the two electrodes comprises transparent semiconducting material. At least one of the two electrodes can comprise graphene operatively connected to the nanowires. The graphene can be deposited by spin coating. The graphene can be optimized for thickness. The optimization of the graphene can be performed through comparisons of a spinning speed in the spin-coating step versus a bending curvature radius of the graphene, spinning speed versus a bending cycle life of the graphene, spinning speed versus a maximum stretching strain of the graphene, or spinning speed versus an optical transmittance of the graphene. A photoresist can be spin-coated on part of the optical device to pattern portions of the electrodes. The photoresist can comprise poly-dimethylsiloxane (PDMS). The electrodes can be patterned with lithography. The electrodes can be etched with ion milling. The electrodes can be etched into micro-stripes by ion milling and shadow masking. A layer of material can be deposited between the semiconducting material and an electrode to form a Schottky barrier. The material layer can be deposited through sputtering. The material layer can comprise gold, or any metallic layer or non-metallic layer with similar electronic properties, i.e. potentially including but not limited to parameters such as work-function, conductivity, and the like. A portion of the optical device can be immersed in polystyrene sulfate (PSS) and poly-dimethylsiloxane (PDMS). The semiconducting material comprises a nanowire array and the portion of the device comprising at least the nanowire array immersed with poly-dimethylsiloxane (PDMS) can be cleaned by oxygen plasma. The nanowires can be functionalized with polystyrene sulfate (PSS). Other polymers and non-polymeric materials with similar electrical properties (i.e. potentially but not limited to those materials with similar ionization potentials and electron affinity values, conductivity, and morphology/mechanical properties) can be used instead of or in addition to the PSS. A portion of the optical device can be conformably covered by a thin layer of Parylene C. Alternatively any variety of polymers, epoxies, and the like that serve as moisture and dielectric barriers can be used. The optical device can be electrical addressed by querying current on the electrodes under a voltage bias.

In another aspect of the disclosure a method of creating an optical device is disclosed. The optical device comprises semiconducting material sandwiched by two electrodes. In an exemplary application of the optical device, it can at least partially comprise a retinal implant. The optical device can at least partially comprise biocompatible materials. It can at least be partially comprised of translucent materials. It can at least partially be comprised of material that allow light to pass through them. It can be deformable. The semiconducting material can exhibit a photon effect. The semiconducting material can comprise material that exhibits a photo-resistance change under illumination. The semiconducting material can comprise nanowires. The nanowires can be arranged in an array pattern. The nanowires can be at least partially comprised of zinc oxide. The optical device can have the zinc oxide deposited through sputtering. The nanowire array can comprise at least one pixel. The at least one pixel can comprise individually clustered groups of the nanowires. At least one of the two electrodes can comprise semi-transparent semiconducting material. At least one of the two electrodes can comprise transparent semiconducting material. At least one of the two electrodes can comprise graphene operatively connected to the nanowires. The graphene can be deposited by spin coating. The graphene can be optimized for thickness. The optimization of the graphene can be performed through comparisons of a spinning speed in the spin-coating step versus a bending curvature radius of the graphene, spinning speed versus a bending cycle life of the graphene, spinning speed versus a maximum stretching strain of the graphene, or spinning speed versus an optical transmittance of the graphene. A photoresist can be spin-coated on part of the optical device to pattern portions of the electrodes. The photoresist can comprise poly-dimethylsiloxane (PDMS). The electrodes can be patterned with lithography. The electrodes can be etched with ion milling. The electrodes can be etched into micro-stripes by ion milling and shadow masking. A layer of material can be deposited between the semiconducting material and an electrode to form a Schottky barrier. The material layer can be deposited through sputtering. The material layer can comprise gold or any metallic layer or non-metallic layer with similar electronic properties, i.e., potentially including but not limited to parameters such as work-function, conductivity, and the like. A portion of the optical device can be immersed in polystyrene sulfate (PSS) and poly-dimethylsiloxane (PDMS. The semiconducting material comprises a nanowire array and the portion of the device comprising at least the nanowire array immersed with poly-dimethylsiloxane (PDMS) can be cleaned by oxygen plasma. The nanowires can be functionalized with polystyrene sulfate (PSS). Other polymers and non-polymeric materials with similar electrical properties (i.e. potentially but not limited to those materials with similar ionization potentials and electron affinity values, conductivity, and morphology/mechanical properties) can be used instead of or in addition to the PSS. A portion of the optical device can be conformably covered by a thin layer of Parylene C. Alternatively any variety of polymers, epoxies, and the like that serve as moisture and dielectric barriers can be used. The optical device can be electrical addressed by querying current on the electrodes under a voltage bias.

In yet another aspect of the disclosure, a biomimetic nanowire optical device configured to be implanted in an eyeball is described. Such a device can comprise an array of ZnO (zinc-oxide based) nanowire piles sandwiched between a top electrode and a bottom electrode, wherein at least one of the top electrode or the bottom electrode comprises a stripe multi-graphene electrode; and a layer of poly-dimethylsiloxane (PDMS) that encapsulates the ZnO nanowire piles, wherein the biomimetic nanowire optical device can be conformably shaped to the dimensions of the eyeball without substantial loss of optical properties. In one aspect, the eyeball is a human eyeball.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other and like reference numerals designate corresponding parts throughout the several views:

FIG. 1, comprising panels 1a , 1a , 1a , 1d, 1e, 1f, and 1g, shows schematic architecture illustrations and images of a nanowire retina with the similar structure of a human retina as its biomimetic replacement.

FIG. 2, comprising panels 2a , 2b , 2c , 2d , 2e , 2f, 2g, 2h, and 2i, illustrates aspects of the flexibility of the nanowire retina which allow for its placement on a 1:1 PDMS human eyeball.

FIG. 3, comprising panels 3a , 3b , and 3c , shows exemplary representative output images of the nanowire retinal prosthesis implanted in the PDMS eyeball system.

FIG. 4, comprising panels 4a, 4b, 4c , 4d, 4e, 4f, 4g, 4h, 4i, and 4j, shows performance evaluation and biological stimulation of the nanowire retina.

FIG. 5, comprising panels 5a , 5b, and 5c , shows an exemplary 3D printer used for manufacturing a 1:1 PDMS human eyeball, the 3D printed molding jig and the 3D printed stage used for casting and holding the hemispherical PDMS eyeball.

FIG. 6, comprising panels 6a and 6b, shows a cross-sectional schematic illustration of an exemplary PDMS eyeball with dimensions and parameters listed, compared with a human eyeball.

FIG. 7, comprising panels 7a, 7b, 7c, 7d, and 7e, shows fabrication and characterization processes of exemplary stripe multi-graphene electrodes.

FIG. 8, comprising panels 8a, 8a, 8c, and 8d, shows the optimization of stripe multi-graphene electrodes with approximately 15 mg/mL graphene solution.

FIG. 9 shows a schematic of the steps for fabricating an exemplary vertical ZnO nanowire cluster pile pixels and nanowire retina.

FIG. 10, comprising panels 10a, 10b, 10c, and 10d, shows an exemplary nanowire retina and transferring to an eyeball.

FIG. 11, comprising panels 11a , 11b, 11c, 11d, and 11e, shows a mapping of projected pixel positions of an exemplary nanowire retina from flat to hemispherical shape. FIG. 12, comprising panels 12a , 12b , 12c , and 12d , shows the strain and the curvilinear transformation of an exemplary nanowire array.

FIG. 13, comprising panels 13a , 13b , and 13c , shows a comparison of the photocurrents and response time of ZnO nanostructured materials with approximately 1 V bias under white light illumination (approximately 10 mW/cm²) while FIG. 13d shows an I-V curve of a single nanowire cluster pile pixel under illumination.

FIG. 14 illustrates a dark current and bad pixel test for an exemplary nanowire retina under approximately 1 V bias, indicating that 63,271 out of 78,010 (approximately 81%) pixels work.

FIG. 15 shows a statistical evaluation on the performance of an exemplary nanowire retina where each graph illustrates the performance under different illumination conditions.

FIG. 16, comprising panels 16a, 16b, 16c, 16d, and 16e, shows that the changing image distance results from adjusting the focus band of an exemplary PDMS eye system.

FIG. 17a shows an optical setup for querying images by an exemplary nanowire retina implanted in a 1:1 PDMS eye system.

FIG. 17b shows grayscale photos printed on transparent films as the objects for imaging while FIG. 17c shows the original photos.

FIG. 18, comprising panels 18a, 18b, 18c, and 18d, shows a first series of images sensed by an exemplary nanowire retina with different resolutions (269×290, 62×72, 20×21, 10×11, respectively) to demonstrate the achievable resolutions for restoring normal vision.

FIG. 19, comprising panels 19a, 19b, 19c, and 19d, shows a second series of images sensed by an exemplary implanted nanowire retina with pixel numbers of 269×290, 62×72, 20×21, 10×11, respectively.

FIG. 20, comprising panels 20a, 20b, 20c, and 20d, shows a third series of images sensed by an exemplary implanted nanowire retina with pixel numbers of 269×290, 62×72, 20×21, 10×11, respectively.

FIG. 21 shows representative queried data of the nanowire retina for the image in FIGS. 19a-19d.

FIG. 22, comprising panels 22a , 22b , 22c , and 22d , shows a) aspects of bio-experimentation for stimulating nerve cells by the nanowire retina based on a live frog. b) The stimulus voltage selection for the stimulating experiments. c) If the stimulating probe does not touch the nerve cells, the frog has no response with electrical stimulating pulses. d) The live frog responds with the electrical stimulating pulses when the probe touches the nerve cells.

FIG. 23 shows the strain distribution as the stimulating probe inserts in the eyeball of a live frog. The results indicate an approximately 5% strain has been introduced. The small strain rate of the eyeball ensures the slight damage on the live frog.

FIG. 24 shows a schematic diagram of an exemplary nanowire retina on a human eyeball working as an implantable nanowire retinal prosthesis for restoring vision.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure.

As used in the specification and the appended claims, the singular forms “a,” “an” and “the” include plural referents unless the context clearly dictates otherwise. Ranges may be expressed herein as from “about” one particular value, and/or to “about” another particular value.

When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

In one aspect of the disclosure, a Nanoelectronic Image System (NETS) is disclosed employing self-assembled semiconducting vertically aligned nanowire (nanowire) arrays as 3D image pixels to sense vision information by photo-resistance change that can be used for imitating a human retina similarly in functions, morphologies, and properties, among other uses. In one aspect of this disclosure, vertical nanowire clustered pile arrays that can be grown with planar process approaches prepare deformable photoreceptors, and multi-graphene stripe electrodes orthogonally connecting the nanowire cluster pile pixels at bottoms and tops function as flexible and transparent interconnects. In one aspect of this disclosure, a thin layer of gold or similar materials can be deposited between the roots of the nanowire clusters and bottom graphene electrodes to form a Schottky barrier, working as a current-gate for reducing photo noise. The entire device can be immersed and protected by materials such as, for example, polystyrene sulfate (PSS) and/or poly-dimethylsiloxane (PDMS) to effectively increase pixel photo-response and device elastomeric durability. The nanowire cluster pile architecture and flexible graphene electrodes yield both active photo sensing components and interconnects flexibility in 3D to withstand stretch and compression with large levels of strains. In one aspect according to this disclosure, a NETS, composed of biocompatible materials, can have the geometric layout to transform into arbitrary curvilinear shapes. Once the NEIS is transformed onto the retina position of an artificial PDMS eye ball (1:1 size ratio with a human eyeball) manufactured by, for example, a 3D printer, it outputs sensed images as a well-established electronic nanowire image sensor (nanowire retina) and has comparable characteristics with a human retina in overall size, pixel size and number, and photo-electric response time.

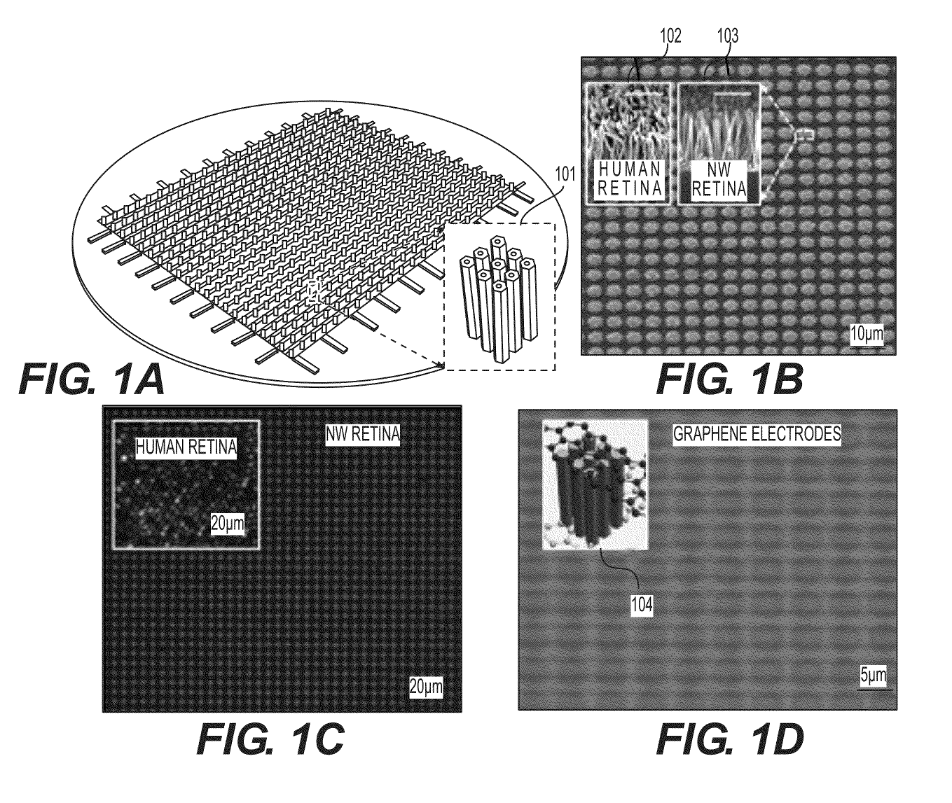

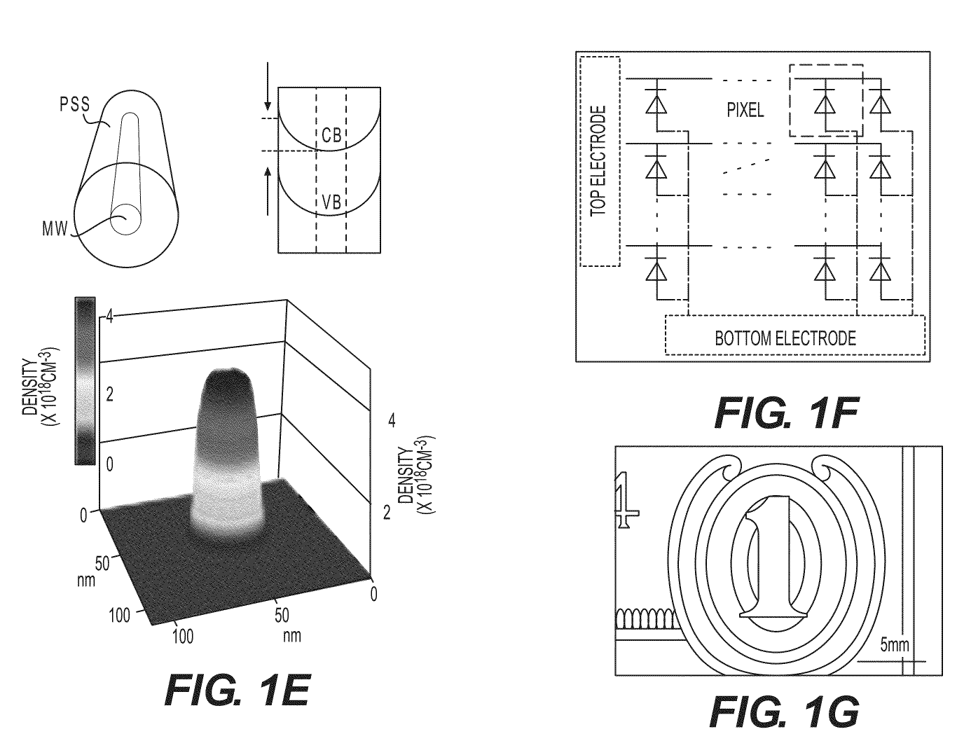

FIG. 1a schematically illustrates the architecture of an exemplary nanowire optical device with a 269×290 matrix pixels fabricated on a flat elastomeric PDMS substrate (having a thickness of approximately 0.3 mm and a size of approximately 2 mm×2 mm). In one aspect, the active component (pixel) can be composed of a cluster pile of light-sensitive photo-resistors such as, for example, vertically grown zinc oxide (ZnO) nanowires (101, FIG. 1a). Stripe shaped bottom and top electrodes orthogonally connect the vertical nanowire cluster piles 101 to electrically address the individual pixels. In one aspect of this disclosure, the entire device of FIG. 1a can be packed in, for example, a thin PDMS film, resulting in a thin, flexible, and transparent retinal prosthesis. This vertical nanowire cluster pile 101 architecture of the pixels is inspired from the structure of human retina. The nanowires can comprise materials other than ZnO as well, including, but not limited to: any semiconducting material, and metal oxide material, carbon nanotubes, and the like. Figure lb shows a scanning electron microscopy (SEM) image of a ZnO nanowire cluster pile arrays grown in a pattern on a PDMS substrate before filling with protective fillers and depositing top electrodes. The left inset 102 of Figure lb shows the microstructure of a human retina and the right inset 103 is the enlarged image of one pixel of an exemplary nanowire retina. The photoreceptor cells (cones and rods) of a human retina stand independently and separately, and keep certain spaces from each other to withstand deforming strains. Thus an element of the design of the nanowire retina can be the use of vertical nanowire cluster piles (averaging approximately 50 nm in diameter and approximately 1.5 μm in height) as the light sensitive pixels, leading to elastic compressibility in individual pixels. The spaces between nanowires and parallel connected multiple nanowire photo-resistors of one pixel allow the pixel to sustain large strains from deformation without damaging the photo-sensing function (in fact, the nanowire itself can withstand more strain compared with its micro/bulk count part, see FIG. 12), similar with the flexible feature of the human retina. (Detailed descriptions of growing vertical nanowire arrays according to the defined pattern as the nanowire retina pixels are discussed further in the Example section.)

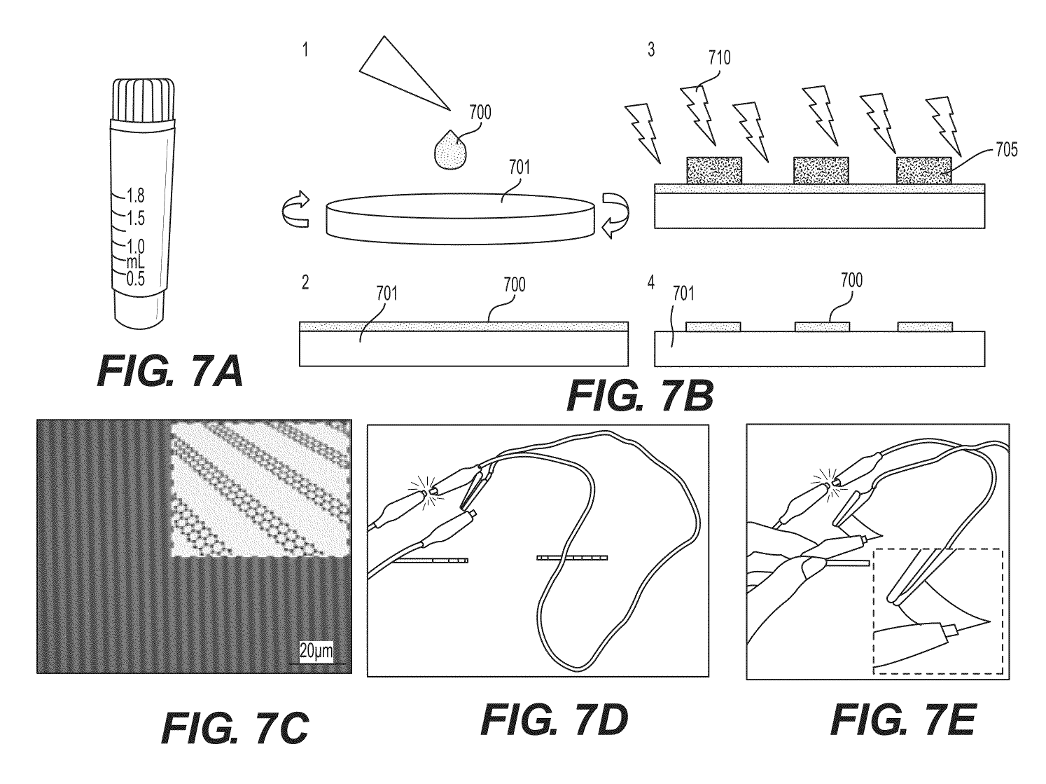

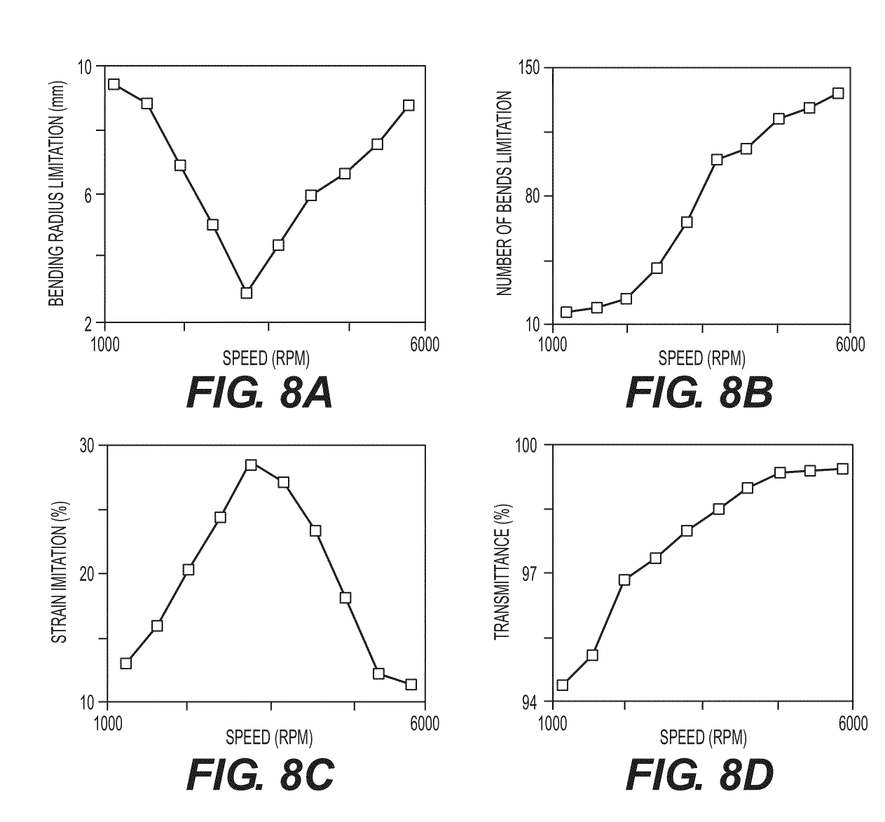

According to one non-limiting aspect of the disclosure, a fabricated nanowire retina has an approximately 2 mm×2 mm working area (similar in size to the macula of the human retina) with a pixel number of 78,010 (269×290, similar to the number of photoreceptor cells in a human retina) as shown in the optical micrograph of FIG. 1c. The pixel size of the exemplary nanowire retina can be controlled at approximately 3.5 μm×3.5 μm (FIG. 1c, approximately 1 μm for neighbor distance), comparable with the size of human visual cells (approximately 3 μm×3 μm for rods, approximately 5 μm×5 μm for cones, inset 104 of FIG. 1d). FIG. 1d is an enlarged top view photograph of an exemplary nanowire retina to reveal the details about the transparent, flexible, and conductive electrodes. These interconnects can be composed of multi-graphene (as the schematic inset of FIG. 1d), consisting of small pieces of single-layer graphene (of size approximately 200 nm to approximately 300 nm). In one aspect, these small graphene pieces can be plated over the entire top and bottom of nanowire retina pixels by spin-coating, and further etched into micro-stripes according to a shadow mask by ion milling (see details in Example section). Graphene, with one atomic thick film morphology, can be transparent, elastic, and can have a high electric conductivity. Thus, graphene can be selected as the electrode material for the nanowire retina. The as-fabricated multi-graphene electrodes preserve transparency (>95%) and high conductivity while allowing large bending deformation strains (the exemplary nanowire retina device can undergo approximately a 4 mm bending curvature radius without noticeable damage; multi-layer slips of the graphene electrodes allow the large strain, and the advantages of multi-graphene electrodes can be found in Example section, see FIGS. 7 and 8).

In one aspect, ZnO nanowire can be selected to form nanowire retina pixels due to its photoelectric properties, which can be improved by functionalizing the nanowire with a film such as a PSS film. The nanowires can however, comprise materials other than ZnO as well, including, but not limited to: any semiconducting material, and metal oxide material, carbon nanotubes, and the like. The PSS can additionally be partially or fully replaced with similar polymers derived from polystyrene containing sulfonic acid or sulfonate functional groups. Other polymers and non-polymeric materials with similar electrical properties (i.e. potentially but not limited to those materials with similar ionization potentials and electron affinity values, conductivity, and morphology/mechanical properties) can be used instead of or in addition to the PSS. The top-left of FIG. 1e shows a schematic of a thin layer (of thickness approximately 3 nm) of PSS coating on ZnO nanowire surface, which can function as a surface oxidization layer to adsorb carriers. The characteristic of oxide-carrier bonds, formed between ZnO nanowires and PSS, can be controlled by the intensity of illumination. Under external illumination, PSS releases the surface bounded electrons. Photocurrent in the nanowires can be induced under external bias as a result of free electrons distributed at the inner surface. This can lead to an improved light-dark photocurrent ratio. Meanwhile, the PSS changing surface energy band states can play a role in shifting the absorption spectra of ZnO nanowires to have a photo response in the visible part of the spectrum. The model of PSS affecting the energy band states of a ZnO nanowire can be shown in the top-right of Figure le. To further understand the interaction between ZnO nanowire electrical carriers and PSS, a numerical simulation of the Meyer-Neldel equations with the finite element method (FEM) can be performed. The distribution of free carriers in the cross-section of the nanowire in the dark can be obtained, as shown in the lower portion of FIG. 1e.

In one exemplary aspect, a thin gold film (approximately 5 nm in thickness) can be formed between the bottom electrodes and the nanowire cluster pile pixels to create a Schottky barrier. The Schottky barrier formed between ZnO nanowires and gold film can lead to improved current-blocking for matrix pixel readout. In various aspects, the layer does not have to be gold, but rather can comprise any metallic layer (e.g. silver, platinum, etc.) or non-metallic layer (indium tin-oxide (ITO), Poly(3,4-ethylenedioxythiophene) Polystyrene sulfonate (PEDOT:PSS), etc.) with similar electronic properties, i.e. potentially including but not limited to parameters such as work-function, conductivity, and the like.

The equivalent circuit diagram of FIG. 1f shows the operation of an exemplary nanowire optical device for image acquisition. In the image sensing process, the light intensity incident on the nanowire pixels can be electrically addressed by querying current by one top electrode and the sequential connections with each bottom electrodes under an approximately 1 V bias. In such an X-Y addressing scheme, individual pixels of the nanowire device can be electrically addressed by one pair of top and bottom electrode combination. The dash line circled region of FIG. 1f is one pixel unit, in which the conducting characteristic of nanowire photo-resistor cluster pile can be modulated by photo illumination. Solid lines in the X-direction and dashed lines in the Y-direction connecting the pixels represent top and bottom stripe electrodes, correspondingly. FIG. 1g shows the transparency (approximately 80%) feature of the approximately 2 mm×2 mm nanowire optical device on one US dollar currency.

In one aspect, the as-fabricated nanowire optical device can be conformably covered by a thin layer of a conformal coating such as, for example, parylene C (Parylene Engineering, San Clemente, Calif.) (of thickness approximately 1 μm) or any other suitable polyxylylene polymer as a protective cover (fabrication details are discussed in the Example section below).

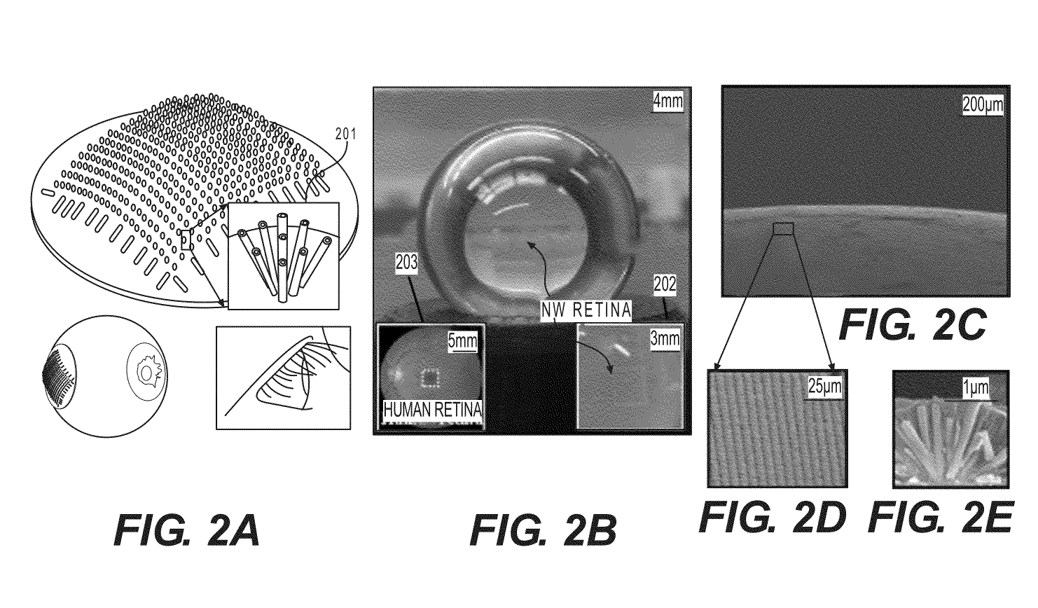

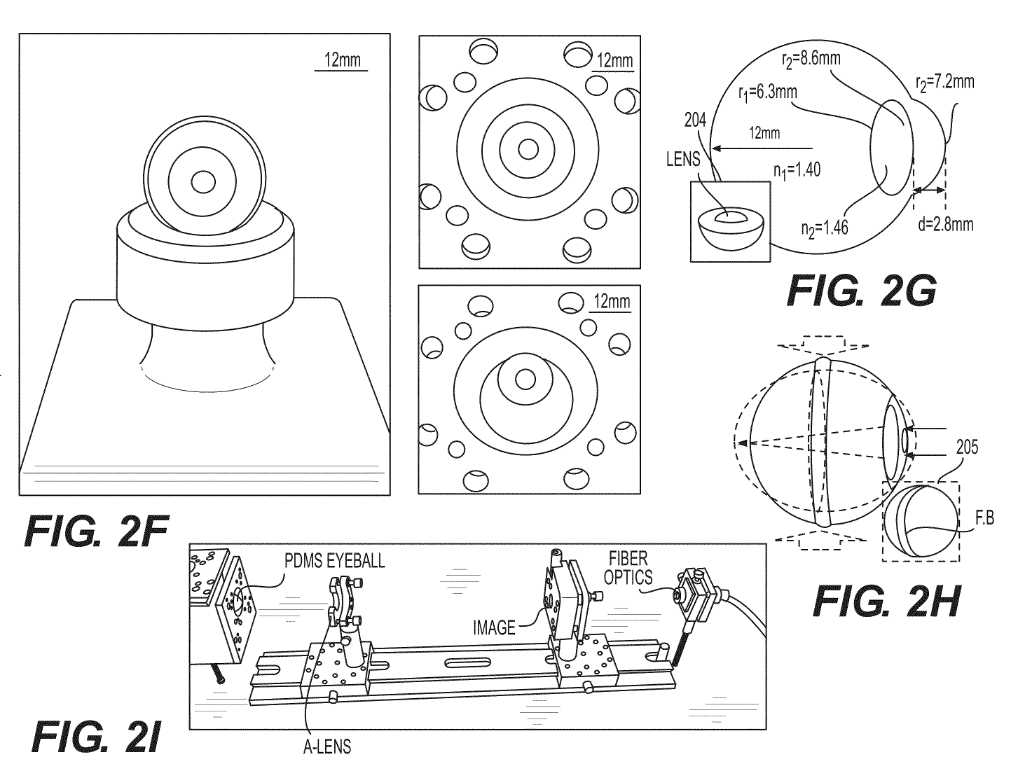

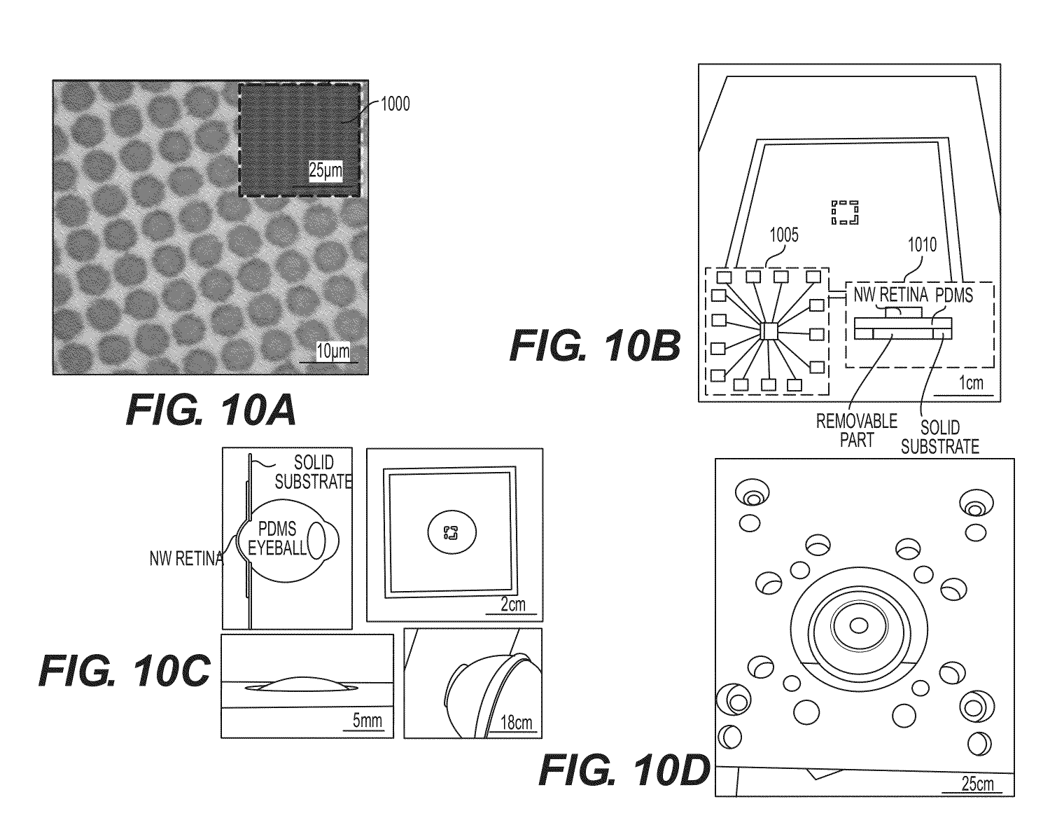

After fabrication, the nanowire optical device can be transferred onto any arbitrary curvilinear surface. FIG. 2a schematically illustrates the nanowire cluster pile structure that enables the flexible feature of the photoreceptor pixels to fit hemispheric shape, like the inner surface of a human eye ball. The slim, vertical nanowires in the cluster pile pixels can accommodate large strains by locally blooming (inset 201 of FIG. 2a) from the original planar position to the adopted arc shape, by mechanics conceptually similar to the spines as a hedgehog rolls up into a ball. This process allows the geometrical transformation of plan-to-sphere accomplished by the entire device without oversized planar strains like previous semi-deformable camera with rigid photodiodes. To demonstrate that the flexible nanowire optical device can perform as retinal prostheses, a 1:1 PDMS human eyeball can be fabricated by 3D printing. FIG. 2b shows a photograph of the PDMS eyeball with a nanowire retina seamlessly transferred/implanted on the back curvilinear surface of the eyeball (the right-inset 202 shows an enlarged image of the nanowire retina) by a ‘printing’ transfer method (see FIG. 10 in the Example section for details). The nanowire retina with, for example, an approximately 2 mm×2 mm active area is placed where the macula is normally located in the human retina (position shown in the left-inset 203 FIG. 2b, the peripherals of the nanowire retina are shown in FIG. 10.). FIG. 2c shows a SEM image illustrating the curvilinear shaped nanowire retina on the PDMS eyeball. FIGS. 2d and 2e show enlarged SEM images of an implanted nanowire retina to illustrate part of the matrix arrays of 269×290 nanowire cluster pile pixels and the details of a deformed pixel, respectively. Nanowire cluster piles can bloom and absorb large strain to avoid functional damage to the pixels.

The process of the nanowire retinal prosthesis that is transferred from planar state to the curvilinear shape according to the back surface of the PDMS eyeball can be simulated by finite element method (FEM) analysis, which can show the detailed strain distribution in individual nanowires, pixels, and the stripe electrodes (See the Example section for details). The simulation indicates that the pixels of the nanowire retina on the hemispherical surface have small projection position variations (approximately 0% to approximately 11%) with their initial positions before the transform, and 8% or smaller position variations for the nanowires in one pixel. In addition, the mechanics model predicts that maximum strains of approximately 0.01% in a ZnO nanowire, approximately 40% in-between spaces of nanowires within one pixel, approximately 40% in the space between pixels, and approximately 30% in multi-graphene electrode for the transform, can be observed.

With 3D printing technology, an exemplary 1:1 PDMS eyeball for demonstrating the performance of the nanowire retina can be formed that is flexible and transparent (greater than approximately 85%, FIG. 2f). The periphery circuit that connects the top and bottom stripe electrodes of the nanowire retina can be operably connected with an A/D board for querying image sensed by the pixels of a nanowire retinal prosthesis. The entire PDMS eyeball and the periphery circuit can be packed in a protective package (right side of FIG. 2f) for mounting on an optical table.

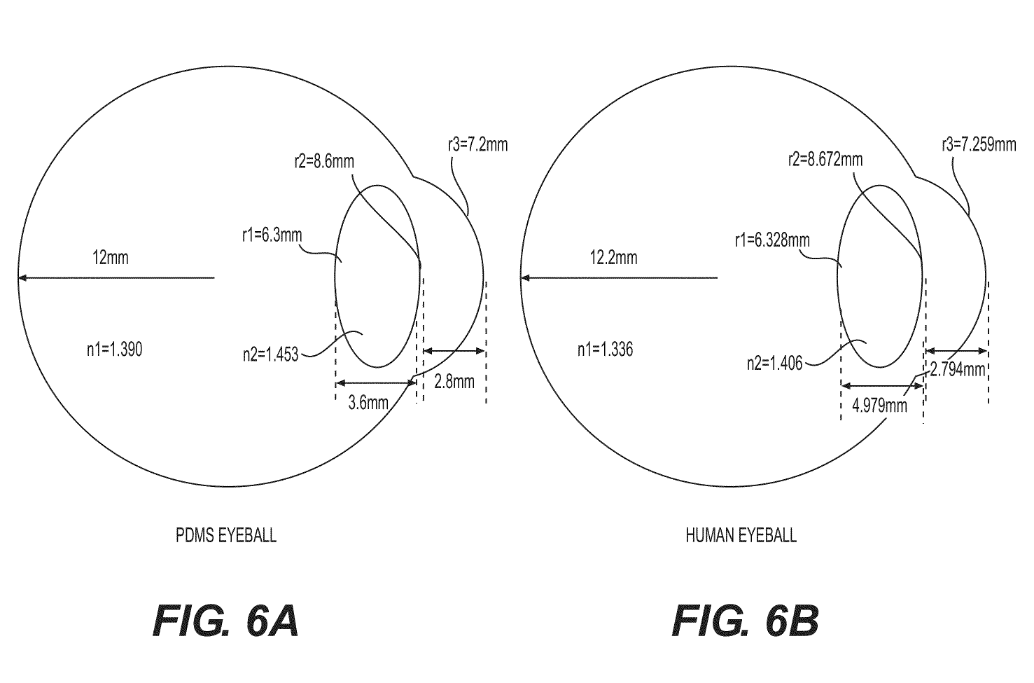

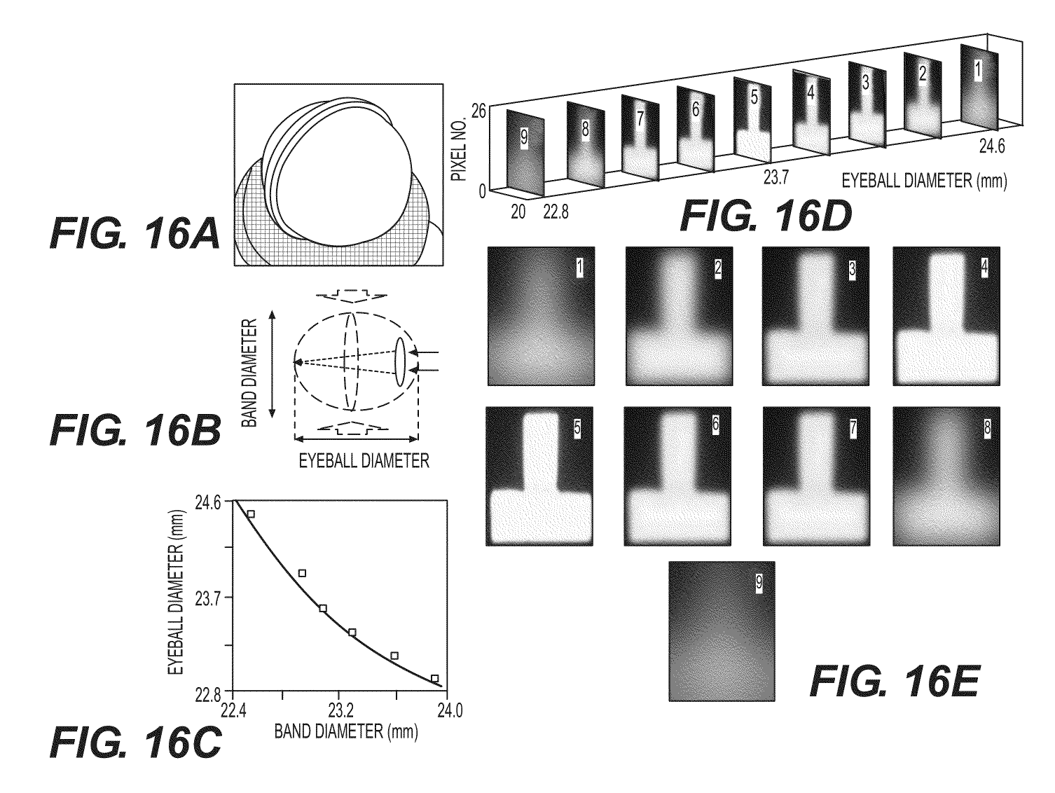

Many parameters of the PDMS eyeball are comparable to a real human eye (FIG. 2g), (FIG. 6 presents the human eyeball system in detail). A glass lens with focal length of approximately 18 mm can be placed in the PDMS eyeball to simulate a human eye lens as the photograph of half-cut PDMS eyeball shown in the inset 204 of FIG. 2g. Because the embedded lens has a fixed focal length, a plastic focus band (inset 205 of FIG. 2h) can be fastened on the middle of the artificial eyeball to change the image distance by adjusting the circumference to form clear image on the nanowire retina for object with various distance, as the schematic FIG. 2h shows (see FIG. 16 for details).

An exemplary optical setup for image acquisition and testing the biomimetic retina is shown in FIG. 2i, including a fiber optic illumination mechanism, an image stage for holding a printed grayscale photo on a transparency film as the object, and a simple auxiliary lens (A-lens) for forming images (additional details of the exemplary system are shown in the Example section).

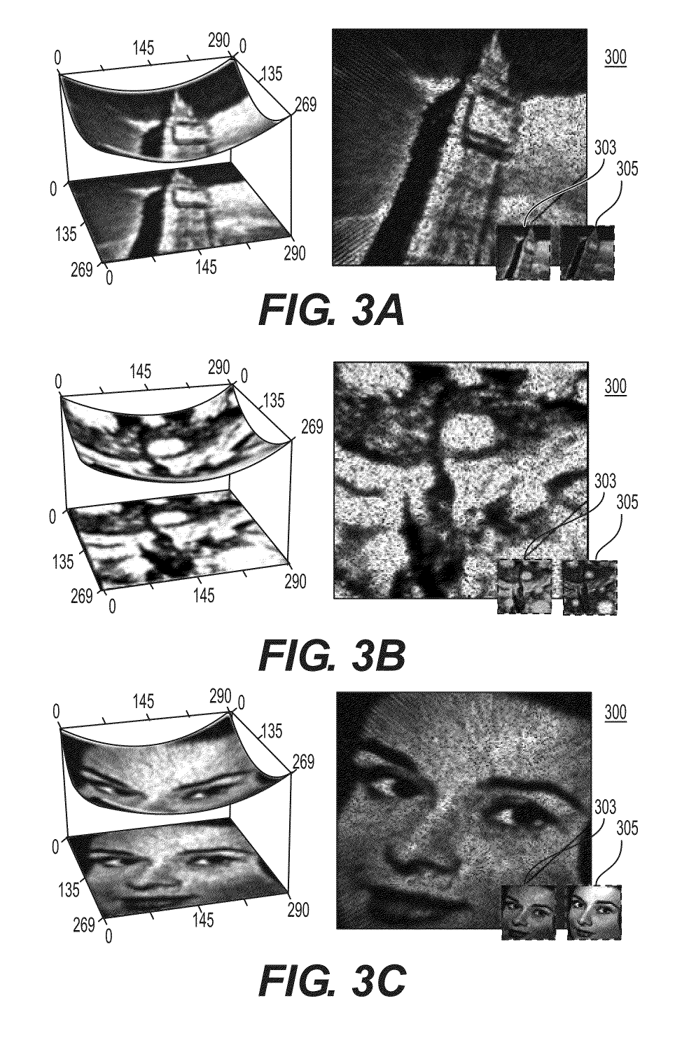

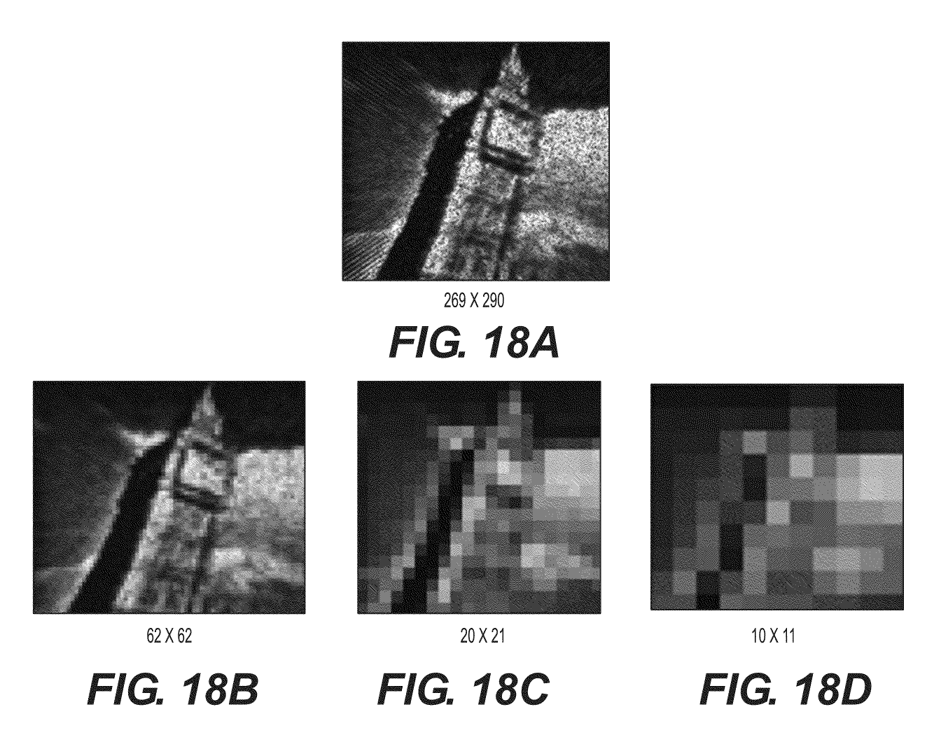

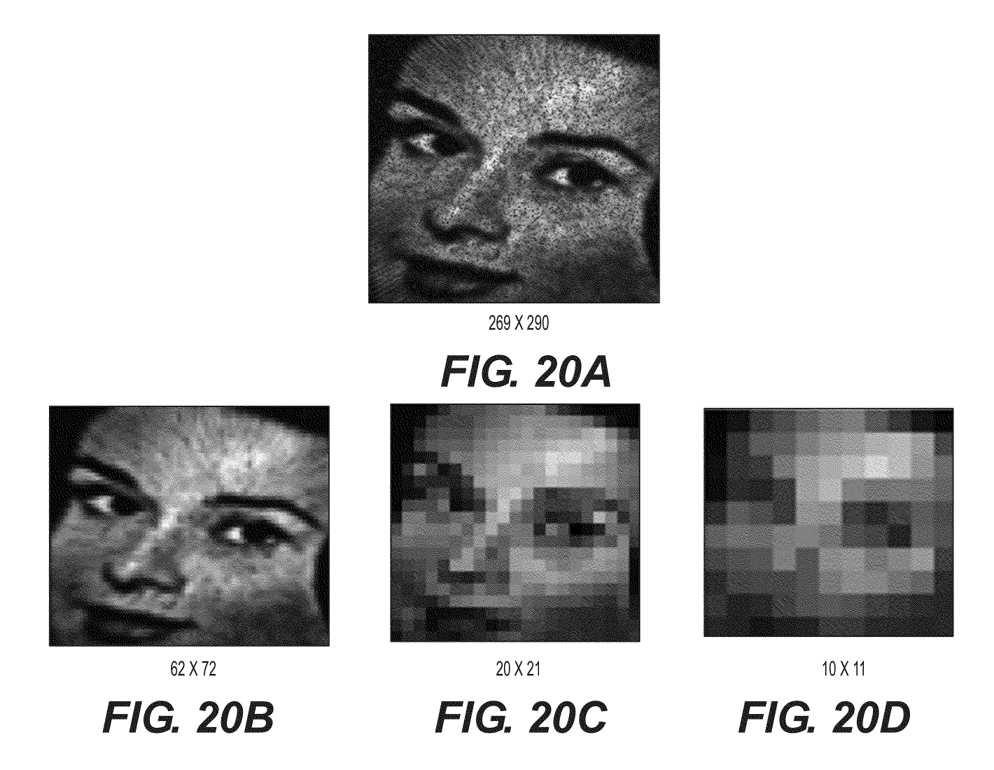

A curvilinear image sensor owns many advantages over planar shaped ones in optical engineering applications, for instance, in obtaining aplanatic images. Thus, the flexible nanowire retina can record better images as it can be transformed onto an artificial or prosthetic eyeball such as the PDMS eyeball described herein, compared with the planar rigid image sensor with the same resolution and optical setup. FIGS. 3a, 3b, and 3c illustrate images recorded by an exemplary nanowire retina implanted on the hemispherical back surface of a PDMS eyeball with the optical setup shown in FIG. 2i. On the left side of FIGS. 3a, 3b, and 3c, the direct image outputs of the nanowire retina for the corresponding images of The Big Ben, The Starry Night, and Audrey Hepburn is shown, which are the grayscale images printed on transparent planar films approximately 1 cm×1 cm in size. The illumination for these objects is white light (approximately 10 mW/cm² light intensity, similar to sunlight illumination) to simulate natural light. Curvilinear shaped images can be the readouts from the nanowire retina pixels on their spatial positions, and the lower images are the projected planar count parts of the arc shape images. Images are recorded by a 269×290 (78010) pixel array of an implanted nanowire retina, which has a similar resolution as compared with a human retina. The images on the right side of FIG. 3 (300) are detailed projected planar images and dash-line-highlighted insets 303 on the left side of 300 are the nanowire retina sensed images resized to compare with the objects (right dash-line-highlighted insets 305). (The comparison and details of different resolution images are also shown in FIGS. 18, 19, and 20.)

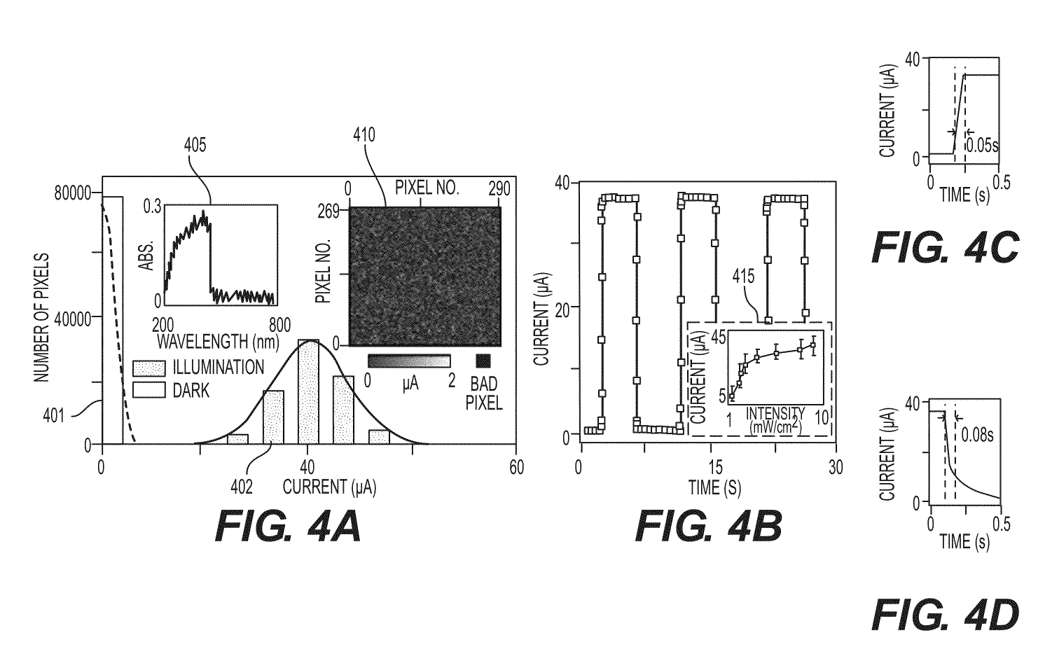

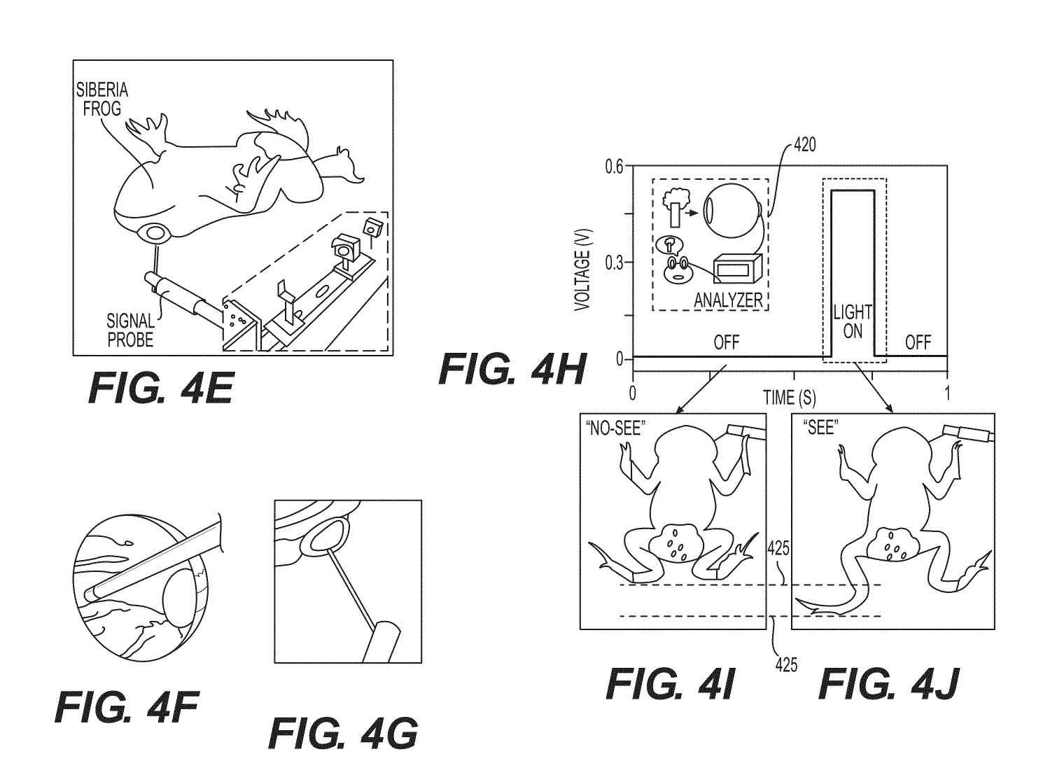

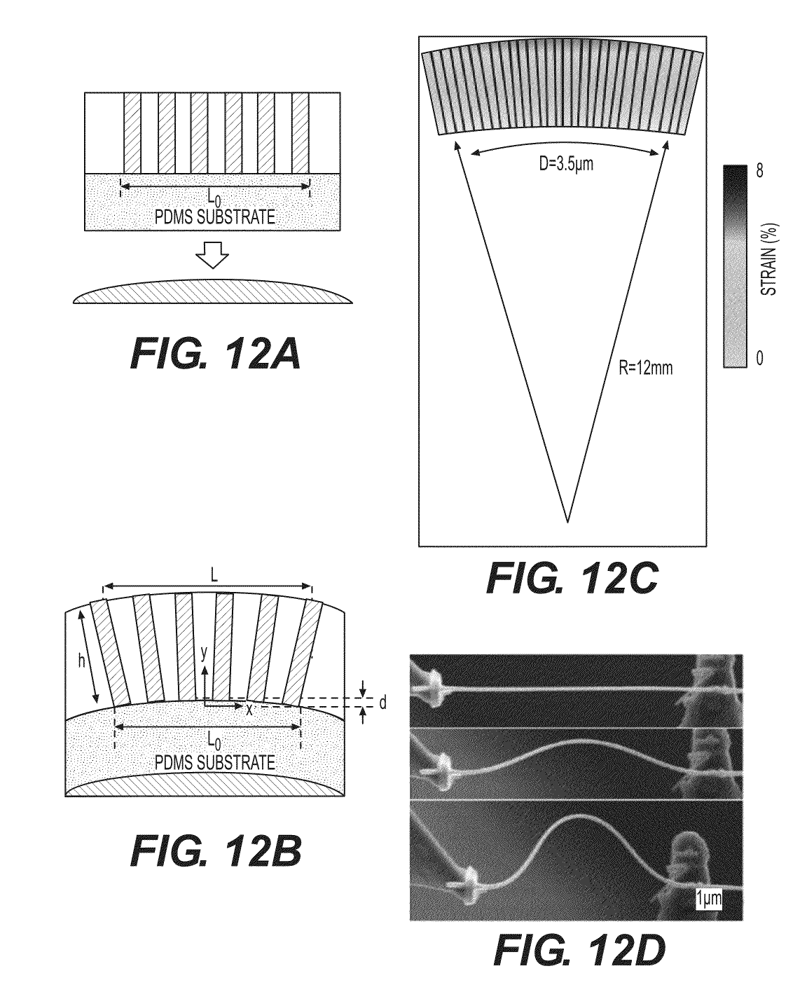

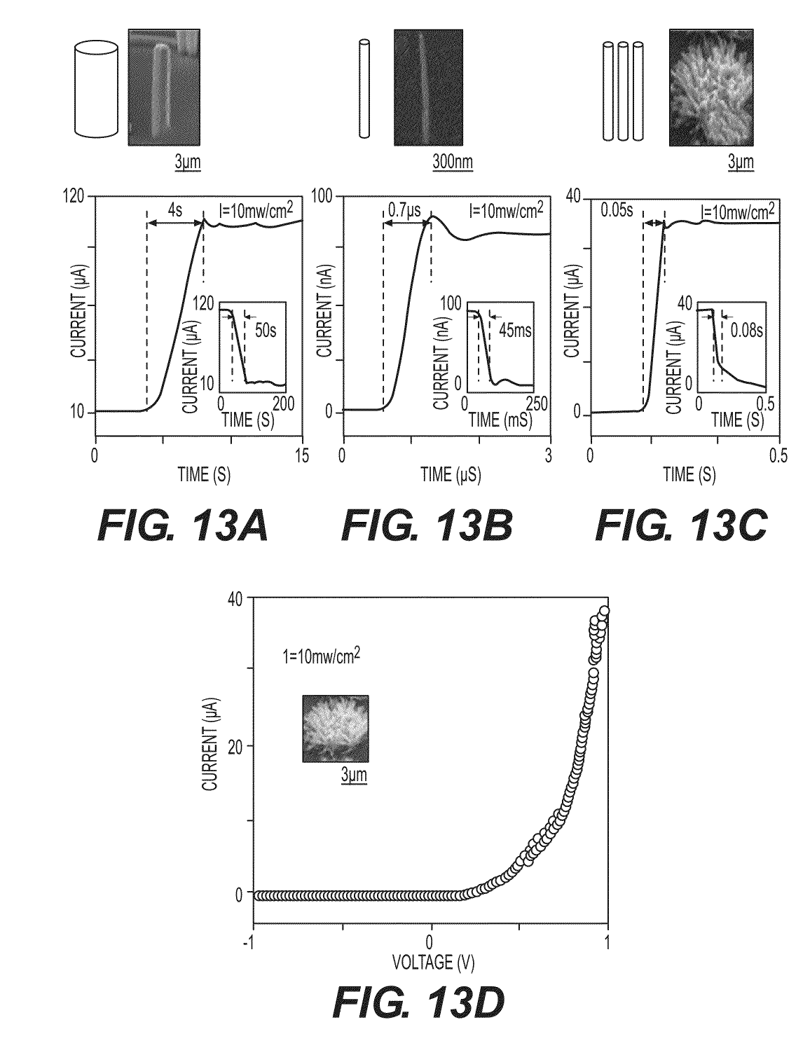

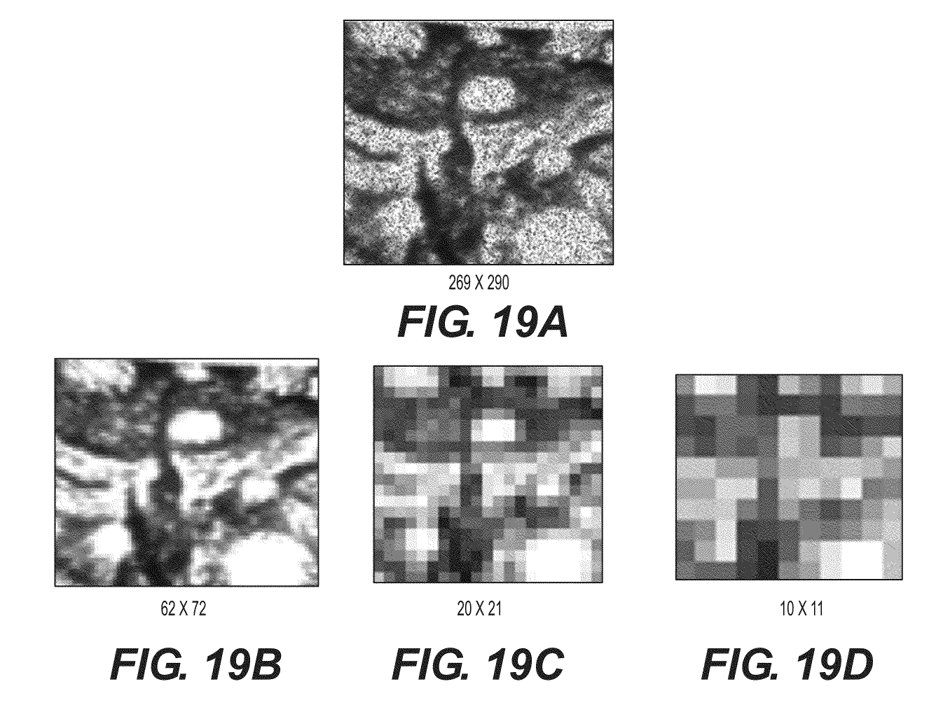

FIG. 4a illustrates a statistical function evaluation of the implanted nanowire retina as it is in the dark and under illumination. The bars 401 and 402 shown in FIG. 4a represent the current output distribution of all pixels of an exemplary optical device as described herein in a dark environment and with light illumination (approximately 10 mW/cm²) under approximately 1V bias, respectively. Under 1V bias, the current values distribute within approximately 0.5±0.7 μA and approximately 40±17 μA for the optical device in dark environment and under illumination, correspondingly, revealing the photoelectrical characteristics of the nanowire cluster pile pixels. (The detailed nanowire retina performance evaluation under various light intensities can be found in FIG. 19). The left inset of FIG. 4a 405 is the nanowire retina pixel absorption spectra, demonstrating a strong light response range from approximately 250 nm to approximately 450 nm. The absorption spectra can be extended to the whole visible wavelength, for example, by doping or surface modifying the nanowires such as ZnO nanowires. The right inset 410 of FIG. 4a shows the metrology mapping of the nanowire retina pixel matrix can correspond to the current outputs of each pixel, revealing 63,271 (approximately 81% of 78,010) pixels of the nanowire retina are functional. The current response of a pixel from the nanowire retina under multi-cycle light illumination is shown in FIG. 4b, revealing stable values of approximately 0.4 μA and approximately 35 μA in the dark and under illumination, respectively. Measured light intensity-dependent currents of one nanowire retina pixel are shown in the inset 415 of FIG. 4b. As a human retinal prosthesis, light response time of the pixel comparable or smaller than that of human photoreceptor cells can be desirable. A ZnO nanowire possesses response time on the order of microseconds, which depends on the structure and height-diameter ratio of the nanowires. To balance the performance between the photo response time and output current value, the cluster pile structured pixel of the nanowire retina can be composed with nanowires of approximately 50 nm in diameter and approximately 1.5 μm in height (details of the photo response time and photo current depending on ZnO nanowire morphology are shown in FIG. 13). FIGS. 4c and 4d show representative measured rising and recovery times of one exemplary nanowire retina pixel, respectively. The total response time of the nanowire retina (approximately 0.13 s) is comparable with that of human retina (approximately 0.1 s to approximately 0.4 s).

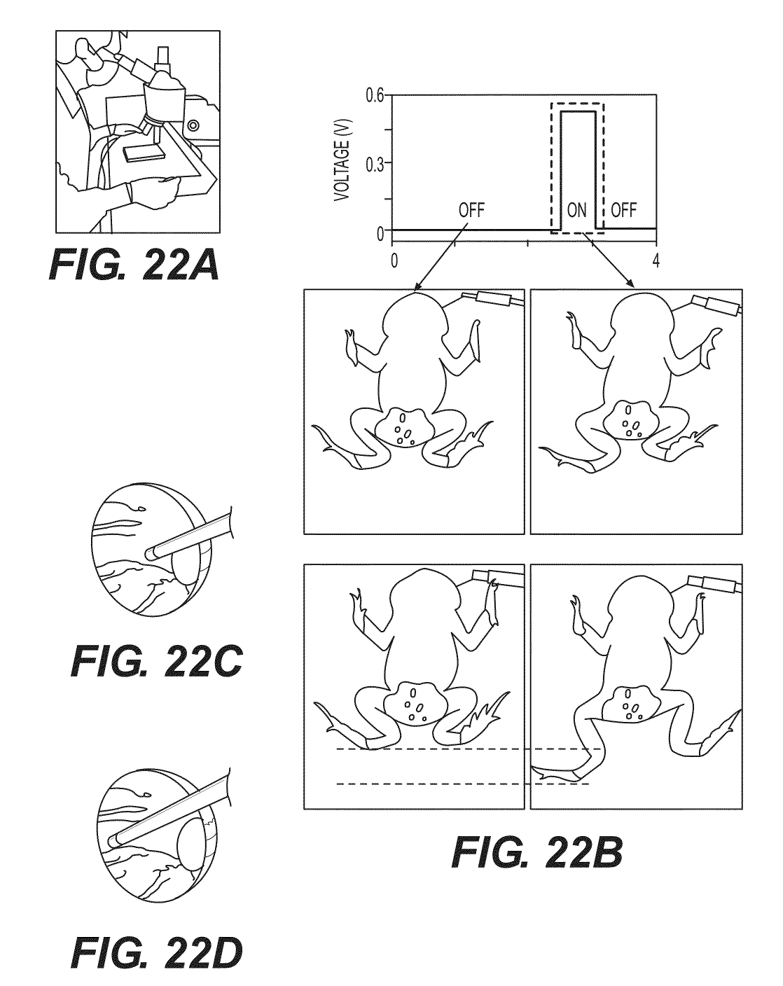

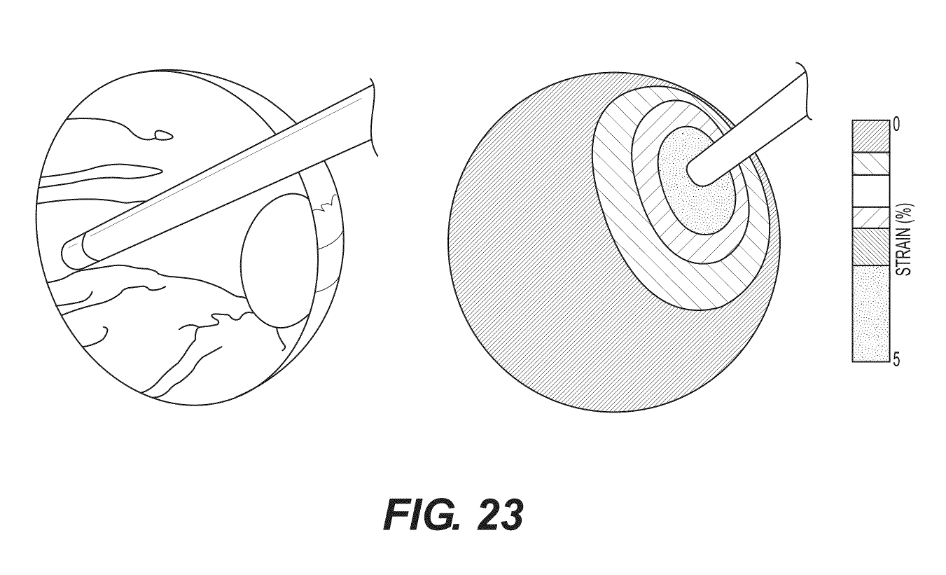

As a retinal prosthesis, the nanowire retina can be used to restore visual function for patients with degenerative retinal diseases by direct implantation without, for example, an external camera system. As an example, a design with functional electrical stimulation, a biological visual stimulation method, has been induced to activate visual nerves, and shows the feasibility of retinal prostheses implantation. A biological experiment can be performed to demonstrate that the electrical signal sensed by the nanowire retina can stimulate the live optical nerve. The active partial implanting surgical trials can be performed on a live Siberia frog (FIG. 4e) with a nanowire retina system, consisting of a signal probe (induce stimulus signal from one nanowire cluster pile pixel), a PDMS eyeball (optical system to form the image), and a data analysis system. The conductive signal probe (approximately 0.1 mm in diameter at the tip), with side insulation to avoid non-visual signal stimulation (FIG. 4f), touches the retinal ganglion cell (RGC) layer of the frog with a low risk of injury (see FIG. 4g and FIG. 23 for details) and delivers an electrical stimulation by the current signal from one nanowire retina pixel. The inset 420 of FIG. 4h illustrates a schematic diagram showing the stimulation mechanism. An image can be projected onto the curvilinear nanowire retina through the PDMS eyeball; the pixels of the nanowire retina sense the higher light intensity and convert it into electrical signals that can be transferred to an analyzer. The analyzer then produces the corresponding voltage signal to stimulate the visual nerves of a frog. Thus, visual signals can be transferred and interpreted by the frog. (A sudden light intensity change sensed by a frog can result in lag stretch due to the natural response of a live amphibian with strong protection awareness. See FIG. 22 for details). FIG. 4h shows the representative stimulating voltage corresponding to a pixel of the nanowire retina. The dash lines 425 display the leg movement of the frog resulted from the stimulation signal received from its visual nerves.

According to the test results, the output voltages were selected as approximately 0.5 V and 0 V, representing illumination and the dark, respectively. More than approximately 80% of the experiment results showed the legs of the frog were in a rest state (FIG. 4i) when the nanowire retina was in the dark, and retracted (FIG. 4j) when the nanowire retina was illuminated (with light of approximately 10 mW/cm²).

### EXAMPLES

The steps, processes and devices described below are to provide a non-limiting examples of applications of an exemplary nanowire optical device as described herein. It is to be appreciated that these are only exemplary applications of the disclosed technology and are not to be limiting in scope or embodiments.

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of the methods and systems. Efforts have been made to ensure accuracy with respect to numbers (e.g., amounts, temperature, etc.), but some errors and deviations should be accounted for.

### Nanowire Retina Preparation

### Soft PDMS Substrate Preparation

1. Clean a silicon wafer (acetone, DI water).
2. Deposit a thin layer of PDMS (approximately 0.3 mm) to the substrate via spin coating method at approximately 500 rpm for approximately 30 s and baking at approximately 85° C. for approximately 15 min.
3. Peel the PDMS film off from the silicon wafer.

### Bottom Electrode Fabrication

4. The graphene (ACS material) is suspended in water under ultrasonication for approximately 30 min, followed by a centrifuge at approximately 2000 rpm for approximately 30 min.
5. The supernatant is dried via an oven at approximately 60° C. Then, the solid is dispersed in water (approximately 15 mg/mL) by ultrasonication for approximately 2 h (FIG. 7a, 700) and spun at approximately 4000 rpm for approximately 15 min onto the PDMS substrates 701 (FIG. 7b1 and FIG. 7b2).
6. Spin-coat photoresist 705 (S1818) on the PDMS substrate and bake at approximately 90° C. for approximately 120 s.
7. Expose the samples using stripe mask with approximately 365 nm UV lithography 710.
8. Develop the exposed sample in developer.
9. Rinse and dry the sample in the oven.
10. Etching the sample with ion milling for approximately 2 min approximately 5 times (FIG. 7b3).
11. Lift-off rest photoresist in acetone (FIG. 7b4).

### Bottom Schottky Contact and Seed Layer Fabrication

12. Clean the processed samples in step 9 (DI water).
13. Pattern photoresist using dots mask with dots alignment on the stripe electrodes (steps 6-9).
14. Deposit approximately 5 nm Au through RF magnetron sputtering.
15. Deposit approximately 10 nm ZnO through RF magnetron sputtering.
16. Lift-off photoresist in acetone.

### Synthesis of Vertical ZnO Nanowire Array

17. Clean the processed samples in step 12 (DI water).
18. The samples are placed into a nutrient solution containing approximately 50 mM zinc nitride (Alfa Aesar) and approximately 50 mM hexamethylenetetramine (HMTA) (Fluka) to obtain nanowire growth at approximately 95° C. for approximately 24 hours.

### Surface Modification and Encapsulation of Vertical ZnO Nanowires Array

19. Clean the processed samples by the process of step 17 (DI water).
20. Spin-coat approximately 3% PSS (in weight) onto the samples at approximately 3000 rpm for approximately 5 min, and bake at approximately 90° C. for approximately 1 min.
21. Spin-coat 17% liquid PDMS (in weight) onto the samples at approximately 2000 rpm for approximately 2 min, and bake at approximately 90° C. for approximately 5 min.
22. Clean sample by oxygen plasma.

### Top Electrode Formation

23. Clean the processed samples as step 19 (DI water).
24. Pattern electrodes using stripes mask according to the bottom stripe electrodes with orthogonal configuration and the crossed areas right cover the tops of nanowire cluster pile pixels (steps 4-11).
25. Perform Parylene C coating (approximately 1 μm thickness)

### Fabrication of a 1:1 PDMS Human Eyeball

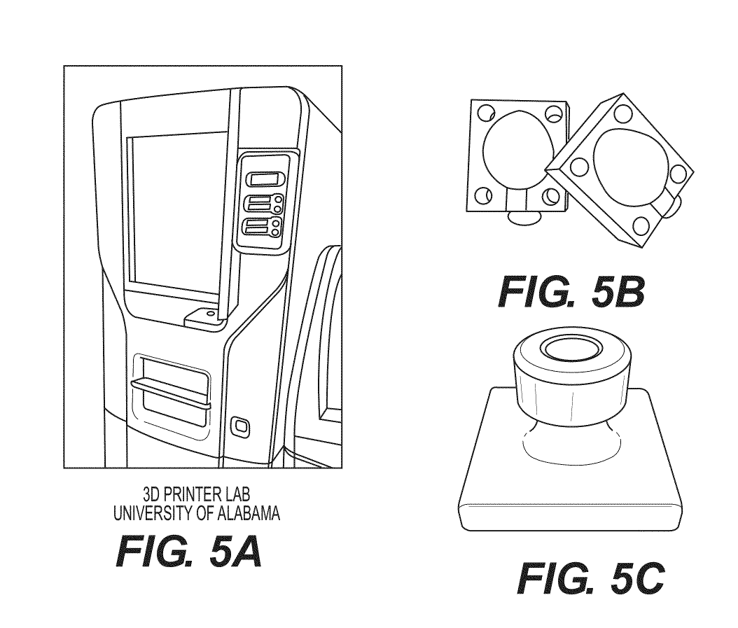

Casting and curing procedures manufactured these 1:1 artificial human eyeballs from PDMS (Sylgard 184). FIG. 5a shows a 3D printer lab in Department of Mechanical Engineering, the University of Alabama, where a highly precise human eyeball mold is printed. FIGS. 5b and 5c show the 3D printed jig (approximately 12 mm radius) used for casting PDMS eyeball and the 3D printed eyeball stage. FIG. 6a provides a cross sectional illustration of an exemplary hemispherical PDMS eyeball with relevant parameters compared to the parameters of a human eyeball (FIG. 6b).

### Stripe Multi-Graphene Electrodes

FIG. 7 shows: a) an optical image of the graphene solution (approximately 15 mg/mL); b) the main steps for the stripe multi-graphene electrodes fabrication by spin coating and ion milling; c) a top-view optical image of the as fabricated stripe multi-graphene electrodes; d) a photograph of the conductive and transparent multi-graphene electrodes; and e) photographs of deformable feature characterization on stripe multi-graphene electrodes.

The multi-graphene electrode (FIG. 7c) with high conductivity, transparency and bendable features (FIGS. 7d and 7e) is employed as the interconnections between nanowire cluster pile pixels. To optimize the performance of a multi-graphene electrode, characterization experiments can be designed and exemplary electrode performance is shown in FIG. 8.

FIG. 8 shows an exemplary multi-graphene electrode: a) spinning speed of coating versus stripe electrodes bending curvature radius; b) bending (approximately 10 mm curvature radius) cycle life of the stripe electrodes as a function of coating spinning speed; c) spinning speed versus maximum stretching strain of the multi-graphene electrode; and d) coating spinning speed versus transmittance of the multi-graphene electrodes. The spinning speed for coating graphene can be chosen at approximately 4,000 rpm to balance the flexibility and transparency.

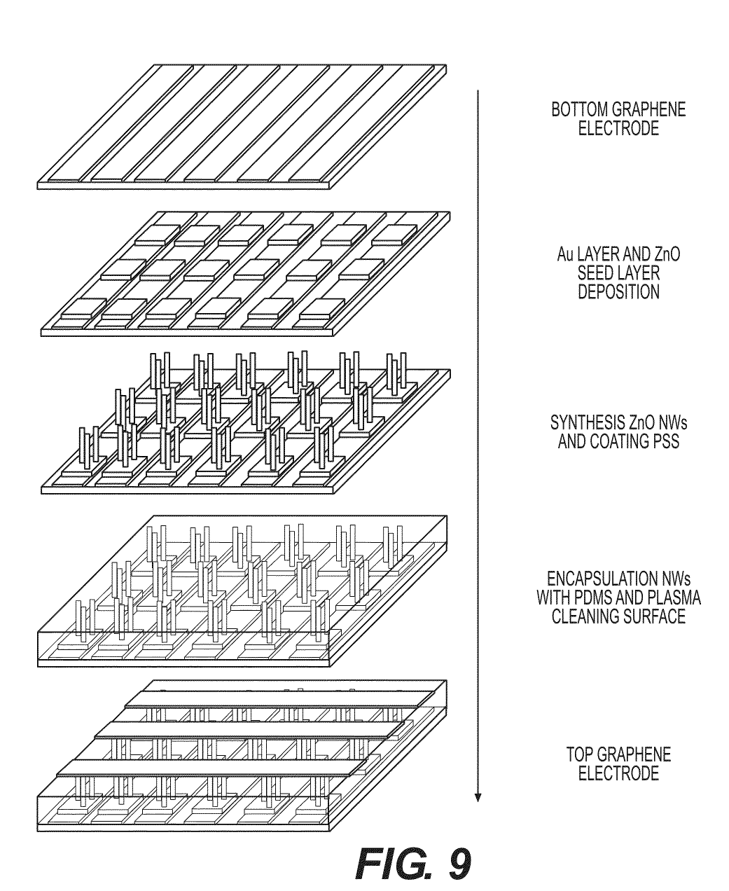

FIG. 9 shows a schematic of the steps for fabricating the exemplary vertical ZnO nanowire cluster pile pixels and nanowire retina. Specifically, the intermediate fabrication steps shown in FIG. 9 comprise: the preparation of the bottom graphene electrode, the deposition of the Au and ZnO seed layers, the synthesis of the ZnO nanowires and their coating with PSS, the encapsulation of the nanowires with PDMS and plasma cleaning of the surface, and finally the deposition of the top graphene electrode. Details for each of these steps can be found herein in various sections of this disclosure including: Soft PDMS Substrate Preparation, Synthesis of Vertical ZnO Nanowire Array, and Surface modification and Encapsulation of Vertical ZnO Nanowires Array in the Nanowire Retina Preparation section.

### Transformation on a Curvilinear Surface and Packaging for Multichannel Measurements

FIG. 10 shows: a) images of nanowire cluster pile pixels before and after top electrodes deposition (inset 1000); b) an image of an exemplary nanowire retina with top and bottom electrodes connected with the inner ends of 269×290 conductive channels where the region outlined by white dashed lines represents a 2 mm×2 mm nanowire retina with 269×290 pixels and the left-inset 1005 shows the schematic diagram of the conductive channels with large outer pads for connecting on a square periphery and the right-inset 1010 shows the schematic diagram cross-section-view of the structure of b; c) show the transfer the nanowire retina onto a PDMS eyeball where the upper left image shows the schematic diagram of the nanowire retina fitting the hemispherical back surface of the PDMS eyeball and the rest are the images of curved nanowire retina on PDMS eyeball, from top-view (upper right), side-view (lower left), and back-view (lower right), respectively; and d) shows an artificial eye system with the nanowire retina implanted in a protective package for querying images.

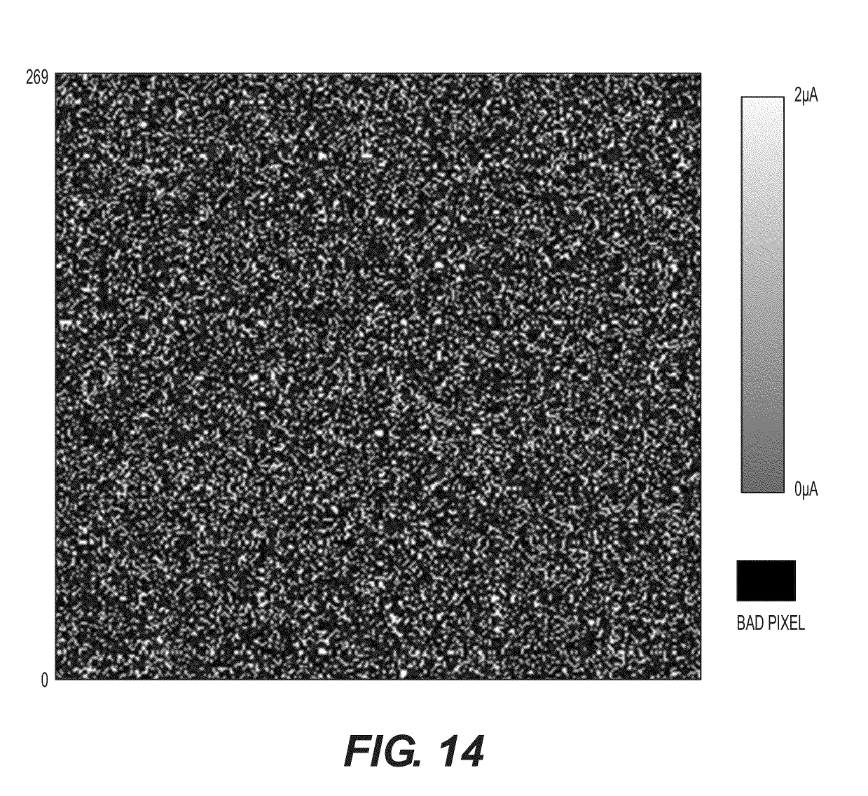

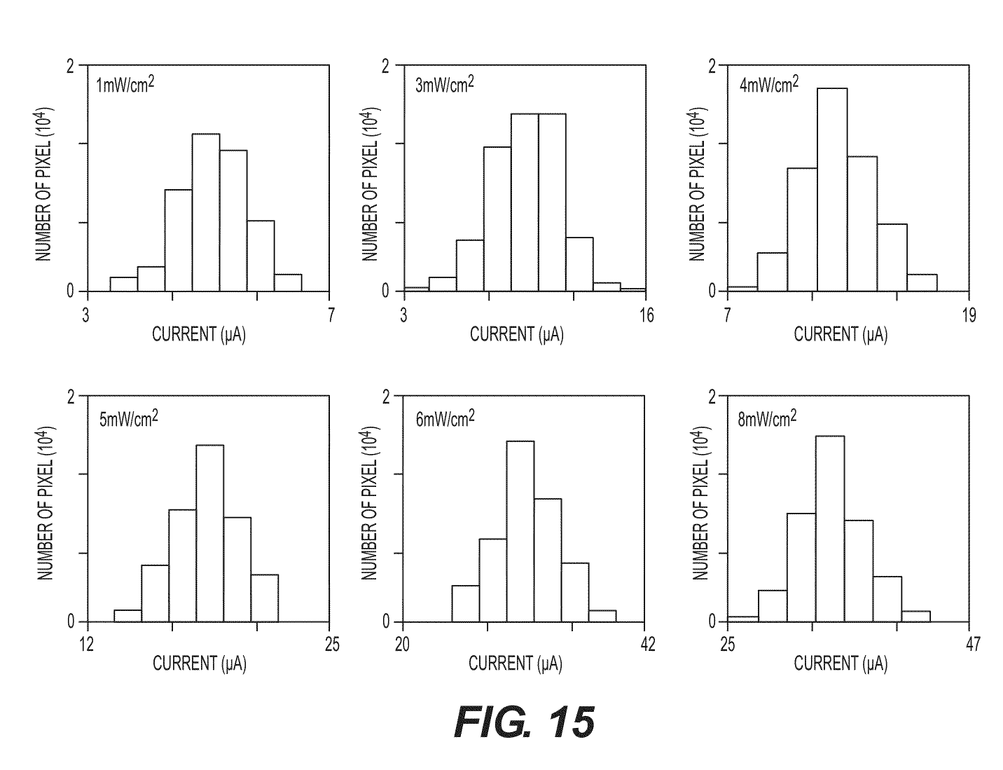

An exemplary nanowire retina can be fabricated on a flat flexible PDMS substrate with flexible photo-sensing pixels and interconnects (FIGS. 10a) and 269×290 electrode channels with inner ends connecting the top and bottom stripe electrodes of the nanowire retina and separated pads at the square periphery as shown in FIG. 10b. Then, a “printing” method can be used to transfer the nanowire retina to the hemispherical back surface of PDMS eyeball, as follows:
1. Attach the nanowire retina on the PDMS substrate with 269×290 conductive channels and connect the top and bottom electrodes with the inner ends of the channels. The PDMS substrate may be formed on a solid bottom substrate.
2. Remove the middle round part (approximately 15 mm in diameter) of the solid substrate without damaging the PDMS substrate.
3. Transfer the as-fabricated system to the PDMS eyeball with the nanowire retina seamlessly fitting the back curvilinear surface through the hole of the solid substrate as FIG. 10c shows. Outer pads of the electrode channels then can be connected to a 600-pin electrical card installed on the protective package board (FIG. 10d). By such configuration, each of the 269×290 pixels can be individually electrically-addressed by iteratively switching two multiplexer. The conductive change of each nanowire retina pixel can be characterized by the current in the circuit under an approximately 1V bias by the mean value within approximately 0.01 s duration through a current meter. The synchronized operations can be controlled by a customized program using, for example, Labview software. Currents corresponding to the pixels that are connected into the measuring circuit can be processed and reconstructed to form the image, fulfilling the image querying process. The dark and illumination currents for the measurement system can be also characterized by this process (see FIG. 14 and FIG. 15). FIG. 14 shows the dark current and bad pixel test for the NW retina under an approximately 1V bias, indicating that 63,271 out of 78,010(˜81%) pixels work. FIG. 15 shows the statistical evaluation on the performance of a NW retina with different illuminating light intensity.
   Mapping Nanowire Pixels from Flat to a Hemispherical Shape

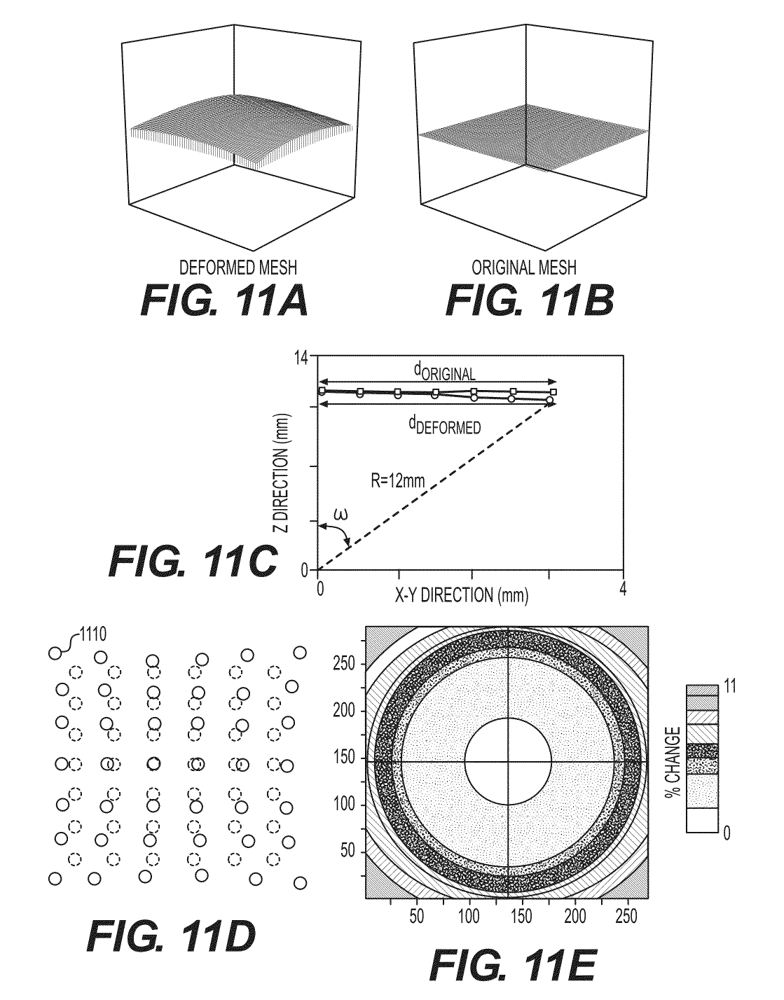

An idea mechanics model, based on FEM analysis, shows how nanowire cluster pile pixels can be mapped from flat shape onto a hemispherical surface. FIG. 11a shows the deformed mesh of a nanowire retina transformed on the back surface of a hemispherical PDMS eyeball, while Figure llb shows the original mesh of the nanowire retina when it is in flat shape before the transform. The projecting position change of center of the nanowire retina is negligible, δ_{center}=0. This can be verified by FEM analysis shown in FIG. 11c. The projecting position change of the nanowire retina then can be expressed by

$${δ}_{circumference} = \frac{ω - sin ω}{ω} .$$

Since each ZnO nanowire cluster pile pixel can be filled with PDMS, the projecting position change in nanowire pixels can be treated as the same as PDMS. For the nanowire retina on a hemispherical surface, the parallel position of each pixel from center of the nanowire retina can be reduced from d_{original} to d_{deformed}.

d_{orignal}=(L_{orignal}+I_{orignal})n

L_{orignal} is the original length between two pixels, I_{orignal} is the original length of one pixel. n is the nth pixel from device center.

And the length of the arc is equal to the d_{original}:

ωR=d_{orignal}=(L_{orignal}+I_{orignal})n

R is the radius of PDMS eyeball.

$$sin ω = \frac{{d}_{deformed}}{R}$$

### Then,

$${δ}_{position} = \frac{{d}_{orignal} - {d}_{deformed}}{{d}_{orignal}} = 1 - \frac{sin [ \frac{( {L}_{orignal} + {l}_{orignal} ) n}{R} ] R}{( {L}_{orignal} + {l}_{orignal} ) n}$$

For the hemispherical PDMS eyeball and L_{orignal}=1 μ, I_{orignal}=3.5 m, the n ranges from 1 to 145.

FIG. 11d shows an image obtained by FEM simulation of the mapping process with original pixel sites as the dash circle indicated for comparison. A schematic diagram (full dots 1110) of projected spatial positions of nanowire pixels on a hemispherical PDMS eyeball is shown. The dash dots represent the original positions of the pixels when the nanowire retina is in a flat shape. FIG. 11e shows the nanowire retina pixel position change rate in the x-y plan projection as it can be transformed from flat into hemispherical shape. These mechanics models indicate approximately 11% changes in the local pitch across the entire area of the nanowire retina.

### Strain Distributions in a Nanowire Retina

FIG. 12 shows: a) a schematic diagram of one nanowire cluster pile pixel before the nanowire retina is transferred to a hemispherical PDMS eyeball back surface; b) a schematic diagram of the deformation distribution in the nanowire pixel after transformation on a hemispherical surface; and c) the simulation of the strain distribution within one pixel where the results indicate up to 8% stain could be observed in the PDMS that is filled in the spaces between nanowires.

Because the Young's modulus of ZnO (approximately 110 GPa) is five orders greater than the Young's modulus of PDMS (2 MPa), the strains in ZnO nanowire are significantly smaller (treated as approximately 0.01%) when the nanowire retina with a structure of nanowires vertically embedded in the PDMS film is transformed into a hemispherical shape. The strains induced in one pixel can be approximately treated as only the strains in the PDMS between nanowires.

The bending energy of a pixel is

$${U}_{1} = \frac{{π}^{4} {Eh}^{3} {d}^{2}}{12 ( 1 - {v}^{2} ) {L}_{0}^{3}}$$

where L₀=3.5 μm is the planar size of the pixel as it is in a flat shape as FIG. 12a shows, d and h are the height of arc of the bended pixel and the thickness of the nanowire retina without bottom PDMS substrate as FIG. 12b shows, E is the Young's modulus of PDMS, v is the Poisson's ratio of PDMS.

And surface energy is

$${U}_{2} = \frac{{EhL}_{0}}{2 ( 1 - {v}^{2} )} {( \frac{{π}^{2} {d}^{2}}{4 {L}_{0}^{2}} - \frac{{L}_{0} - L}{{L}_{0}} )}^{2} .$$

L is the middle planar length of a bended pixel as FIG. 12b shows.

With minimized energy, we can have

$$\frac{\partial {U}_{1} + {U}_{2}}{\partial d} = 0 ,$$

the height of arc d can be obtained as

$$d = \frac{2 {L}_{0}}{π} \sqrt{\frac{{L}_{0} - {L}_{1}}{{L}_{0}} - \frac{{π}^{2} {h}^{2}}{3 {L}_{0}^{2}}} ,$$

and strain distributions in the nanowire pixels is

$$ɛ = \frac{{π}^{2} {h}^{2}}{3 {L}_{0}^{2}} ,$$

FIG. 12c shows the strain distribution of one nanowire pixel with a 12×12 nanowires (the average nanowire number in one pixel) transferred to a hemispherical back surface of the PDMS eyeball calculated by the mechanics model above. These mechanics model indicates up to approximately 8% strain in the PDMS which can be filled in the spaces of the nanowires. Additionally, the large-bending curvature of ZnO nanowires (approximately 50 nm in diameter) in previous literature (FIG. 12d) proves that a ZnO nanowire itself can withstand a large deformation.

The Photoelectric Property Change with Nanowire Pixel Structure.

With the variance of nano-confinement strength, the photoelectric property of ZnO nanostructures will be different. To optimize the performance of the artificial retina pixel, characterization of the photoelectric property of ZnO nanostructures with different size and morphology can be performed. The photocurrents can be measured on the ZnO nanomaterials with approximately 1V bias under white light illumination of approximately 10 m W/cm². FIG. 13a shows a single ZnO nanowire with large radius (approximately 450 nm) has large current (approximately 110 μA) but slow response time (approximately 4 s rising and approximately 60 s recovery). FIG. 13b shows that a single ZnO nanowire with a small radius (approximately 25 nm) has fast response time (approximately 0.7 μs rising and approximately 45 ms recovery) but small response current (approximately 80 nA). FIG. 13c shows a ZnO nanowire cluster pile with small nanowire radius (approximately 25 nm) has intermediate response time (approximately 0.05 s rising and approximately 0.08 s recovery) and photocurrent (approximately 40 μA). With the comparison, ZnO nanowire cluster pile with small nanowire radius (approximately 25 nm) was chosen as the photoreceptor pixel for an exemplary nanowire retina with relative large response current (approximately 40 approximately μA) and fast response time (approximately 0.05 s rising and approximately 0.08 s recovery). The photoelectric response of a representative individual nanowire retina pixel is shown in FIG. 13d.

### Image Distance Adjusting Procedure of the PDMS Eyeball

FIG. 16a shows the photograph of the 1:1 PDMS eyeball with a focus band for image distance adjustment. FIG. 16b presents schematic principle of changing PDMS eyeball image distance by adjusting the focus band for objects with different distance to form clear image on the nanowire retina. FIG. 16c shows the experimental curve of the transverse diameter of the PDMS eyeball as a function of focus band diameter. FIGS. 16d and 16e show the recorded images of an object letter “T” with approximately 18 mm distance from the PDMS eye acquired by 26×20 pixels of the nanowire retina. The images are collected by the PDMS eye system with transverse diameter ranging from approximately 22.8 mm (image 9, left) to approximately 24.6 mm (image 1, right). The right image distance is close to approximately 23.7 mm.

### Optical Setup and Image Resolution

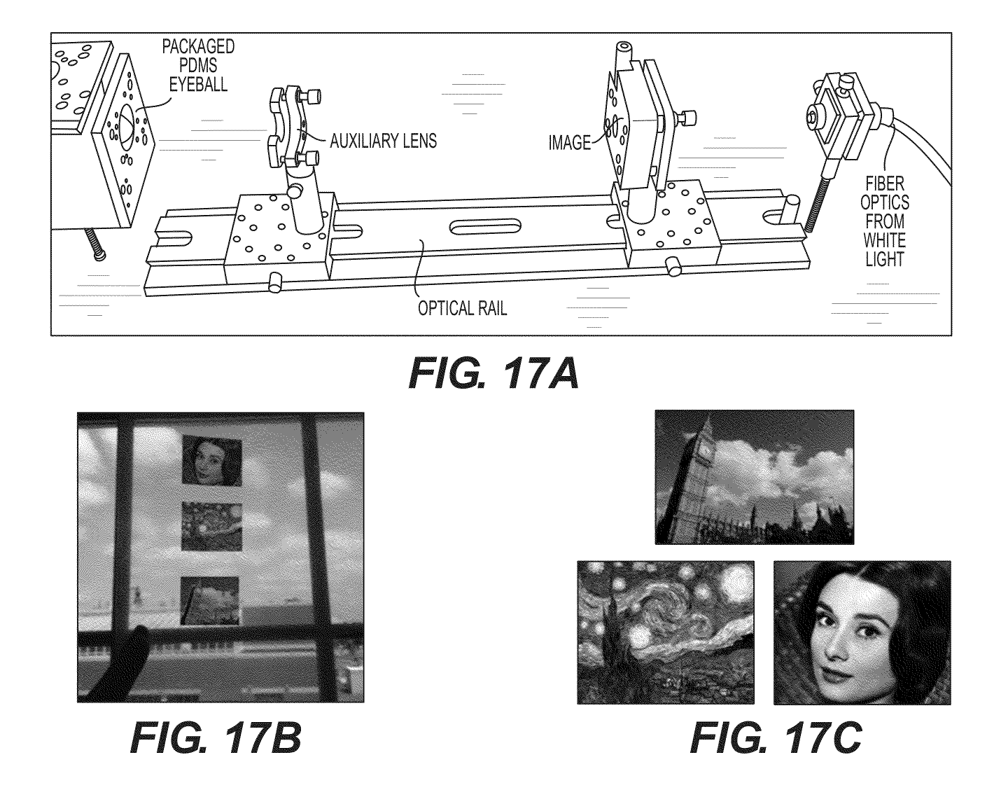

FIG. 17a demonstrates the optical setup used for the nanowire retina sensing images.

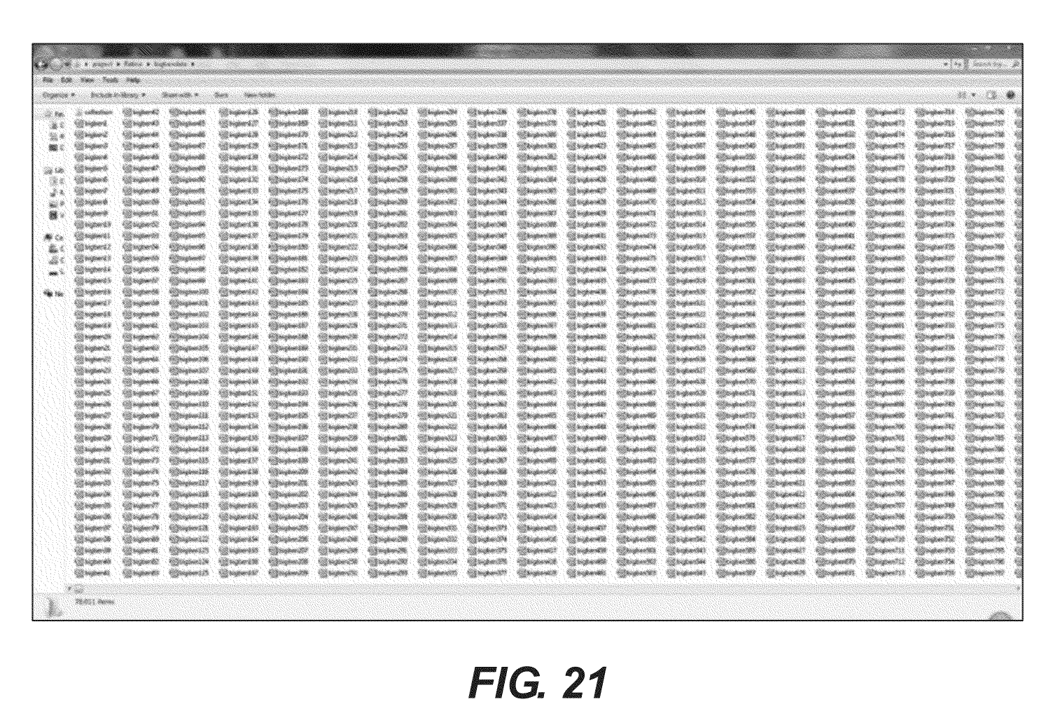

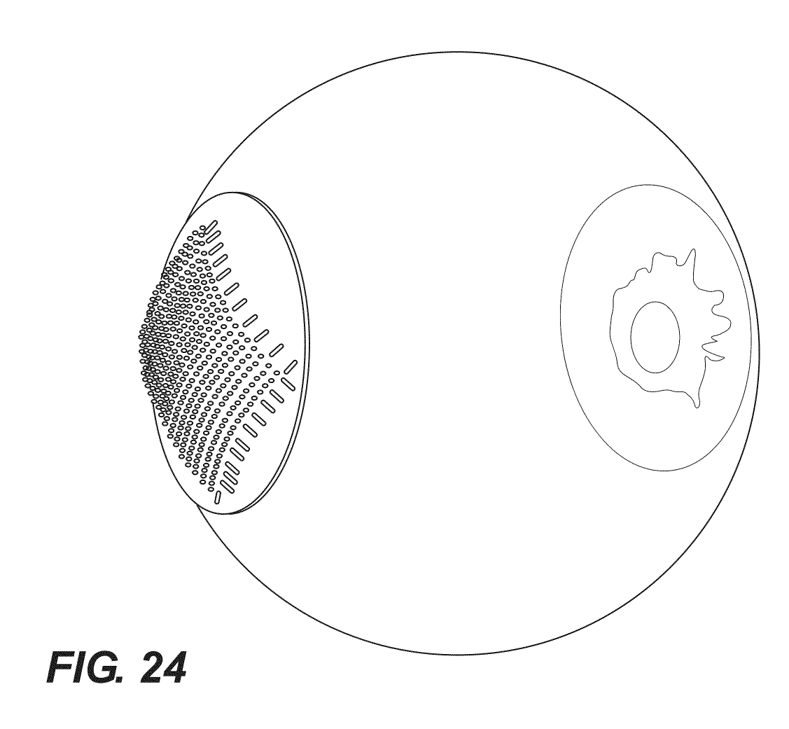

Through optical fiber and an integrated auxiliary lens, white light illuminates the grayscale photos printed a transparency film (size of approximately 2 cm×2 cm) as the objects (FIG. 17b, FIG. 17c are the original photos). The objects form images on the PDMS eyeball nanowire retina through a convex lens. FIGS. 18, 19, and 20 are the corresponding images that were sensed by the nanowire retina with different resolution by nanowire retina pixel number of 269×290, 62×72, 20×21, 10×11, respectively. The higher resolution images can reveal more details, indicating higher resolution. FIG. 21 shows a representative high resolution image data with the full pixels (269×290) of the implanted nanowire retina. FIG. 24 shows a schematic diagram of the nanowire retina on a human eyeball working as an implantable NW retinal prosthesis for restoring vision.

### Bio-Experimentation Procedure

1. A Siberia frog is chosen for nanowire retina implant experiments.
2. Place a piece of ether cotton into the box where the live frog is held. Approximately 3 min later, frog achieves the effect of anesthesia.
3. Take the frog out and put it on a lab board.
4. Fix the main body of the frog on the board.
5. Set a probe on a cantilever of the probe station.
6. Adjust the cantilever (FIG. 22a) to insert the probe into the eye from the side of the pupil and touch the nerve cells, to limit damage to the frog eye system (FIG. 23).
7. With the probe connected to a stimulus voltage generator, fine adjust the inserting depth of the probe to find the position of the nerve cells by stimulus signal (0.5V, FIG. 22b). (If probe does not touch the nerve cells, the frog has no response as the case in FIG. 22c. If probe does touch the nerve cells, the frog has response as FIG. 22d.)
8. Connect the analyzer with the PDMS eye system after locating the right position for the probe touching with the nerve cells.
9. Repeat the optical nerves response experiments for statistical analysis.

Greater than approximately 80% of 30 stimulation experiments show the leg extension under the stimulation from one pixel of the nanowire retina.

### CONCLUSION

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications may be referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. An optical device comprising:
a plurality of substrates of semiconducting material, collectively, forming a sensor array, wherein each of the substrates of plurality of semiconducting material is operatively connected to and sandwiched between two flexible electrodes.

2. The optical device of claim 1, wherein each of the substrates of semiconductor material forms a cluster pile of the semiconducting material, and wherein the substrate exhibits a photon effect under illumination.

3. The optical device of claim 1, wherein each of the substrates of semiconductor material form a cluster pile of the semiconducting material, wherein the substrate exhibits a photo-resistance change under illumination.

4. The optical device of claim 1, wherein each of the substrates of semiconducting material comprises nanowires arranged in a vertically aligned array pattern.

5. The optical device of claim 4, wherein the nanowire array comprises at least one pixel.

6. The optical device of claim 1, wherein at least one of the two electrodes comprises semi-transparent semiconducting material.

7. The optical device of claim 1, wherein at least one of the two electrodes comprises transparent semiconducting material.

8. The optical device of claim 1, wherein at least one of the two electrodes comprises graphene operatively connected to the nanowires.

9. The optical device of claim 1, comprising a Schottky barrier formed between semiconducting material and at least one of the two electrodes.

10. The optical device of claim 1, wherein each of the substrates is electrically addressable by querying current on the electrodes under a voltage bias.

11. A method of fabricating an optical device comprising a plurality of substrates of semiconducting material, collectively, forming a sensor array, wherein each of the substrates of plurality of semiconducting material is operatively connected to and sandwiched by between two flexible electrodes, the method comprising:
depositing, via sputtering, a semiconductor material to form a cluster pile of the semiconducting material; and
depositing, via spin coating, to form the electrodes.

12. The method of claim 11, wherein the deposition, via the spin coating, comprises an optimization step selected from the group of:
comparing a spinning speed used during the spin-coating to a bending curvature radius of a material comprising the electrodes,
comparing the spinning speed to a bending cycle life of the electrode material,
comparing the spinning speed to a maximum stretching strain of the electrode material, and
comparing the spinning speed to an optical transmittance of the electrode material.

13. The method of claim 11, comprising: spin-coating a photoresist on part of the optical device to pattern portions of the electrodes.

14. The method of claim 13, comprising: patterning, via lithography, the electrodes.

15. The method of claim 13, comprising: etching, via ion milling, the electrodes into micro-stripes.

16. The method of claim 11, comprising: depositing a layer of material between the semiconducting material and an electrode to form a Schottky barrier.

17. The method of claim 11, comprising: sputtering a layer of material between the semiconducting material and an electrode to form a Schottky barrier.

18. The method of claim 11, comprising: cleaning, via oxygen plasma, the substrates of semiconducting material.

19. A biomimetic nanowire optical device configured to be implanted in an eyeball, said device comprising:
an array of Zinc-Oxide based nanowire piles sandwiched between a top electrode and a bottom electrode, wherein at least one of the top electrode or the bottom electrode comprises a stripe multi-graphene electrode; and
a layer of poly-dimethylsiloxane (PDMS) that encapsulates the ZnO nanowire piles,
wherein the biomimetic nanowire optical device can be conformably shaped to the dimensions of the eyeball without substantial loss of optical properties.

20. The biomimetic optical device of claim 19, wherein the eyeball is a human eyeball.
